(19) United States
(12) Patent Application Publication
(10) Pub. No.: US 2026/0201438 A1
(43) Pub. Date: Jul. 16, 2026

(54) DIRECT-FROM-SAMPLE ANTIBIOTIC SUSCEPTIBILITY TEST FOR COMPLICATED URINARY TRACT INFECTIONS

(71) Applicant: Fundamental Solutions Corporation, Beaufort, SC (US)

(72) Inventors: Joseph D. Kittle, JR., Beaufort, SC (US); Joel Lwande, Beaufort, SC (US)

(21) Appl. No.: 19/562,149

(22) Filed: Mar. 10, 2026

Related U.S. Application Data
(63) Continuation-in-part of application No. 17/965,207, filed on Oct. 13, 2022.
(63) Continuation-in-part of application No. 18/907,908, filed on Oct. 7, 2024.
(60) Provisional application No. 63/880,032, filed on Sep. 11, 2025.

(51) Int. Cl. C12Q 1/18; G01N 21/64; G01N 33/569; G01N 33/68

(52) U.S. Cl. CPC C12Q 1/18; G01N 21/6428; G01N 33/56911; G01N 33/6812; G01N 2021/6439

(57) ABSTRACT

A direct-from-sample antimicrobial susceptibility testing system that includes a biological sample that is processed to capture microorganisms present in the sample; an acclimatization medium into which the microorganisms are transferred that activates protein biosynthesis in the microorganisms; a library of antimicrobials at predetermined concentrations to which the sample is exposed, wherein the exposure of the microorganisms to the library of antimicrobials either kills or blocks protein biosynthesis in the microorganisms that are sensitive to one or more of the antimicrobials at one or more of the predetermined concentrations; a non-canonical amino acid that is incorporated into newly biosynthesized proteins in the microorganisms that initially survive exposure to the antimicrobials thereby labeling the newly biosynthesized proteins; and a detectable tag configured to attach to the non-canonical amino acid labels in the newly biosynthesized proteins, wherein tagging the labeled proteins with the detectable tag creates an amount of detectable signal.

### CROSS REFERENCE TO RELATED APPLICATIONS

This patent application is a continuation-in-part of U.S. patent application Ser. No. 17/965,207 filed on Oct. 13, 2022 and entitled “Antibiotic Susceptibility Test”; and U.S. patent application Ser. No. 18/907,908 filed on Oct. 7, 2024, and entitled “System and Method for Antimicrobial Susceptibility Testing”, the disclosures of which are hereby incorporated by reference herein their its entirety and made part of the present U.S. utility patent application for all purposes. This patent application also claims the benefit of U.S. Provisional Patent Application Ser. No. 63/880,032 filed on Sep. 11, 2025, and entitled “A Rapid and Scalable Bacterial Labeling Method for Phenotypic Antimicrobial Susceptibility Testing”, the disclosure of which is hereby incorporated by reference herein in its entirety and made part of the present U.S. utility patent application for all purposes.

### BACKGROUND

The disclosed inventive subject matter relates in general to systems, devices, and methods for use in diagnosing and treating infectious disease, and more specifically to a rapid and highly accurate antimicrobial or antibiotic susceptibility test for determining the susceptibility of bacteria and other microorganisms to various antibiotics or antimicrobials for treating simple urinary tract infections and complicated urinary tract infections, in particular.

A urinary tract infection (UTI) is an infection that affects a part of the urinary tract which includes the kidneys, ureters, bladder, and the urethra. Symptoms from a lower urinary tract infection include suprapubic pain, painful urination, frequency and urgency of urination despite having an empty bladder. Symptoms of a kidney infection are more systemic and include fever or flank pain usually in addition to the symptoms of a lower UTI. UTIs are among the most common causes of sepsis in hospitalized patients. UTIs have a wide variety of presentations and some UTIs can be managed with outpatient antibiotics and carry a reassuring clinical course with an almost universally good outcome. On the other end of the spectrum, florid urosepsis in a comorbid patient can be fatal. UTIs can also be complicated by various risk factors leading to treatment failure, repeat infections, or significant morbidity and mortality with a poor outcome.

A simple UTI (or cystitis) is a urinary tract infection caused by bacteria that are known (in a clinical context) to be antibiotic-susceptible and that is not associated with treatment failure or poor outcomes. Typically, this is an infection in an afebrile, nonpregnant, immune-competent female patient. A complicated UTI is any UTI other than a simple UTI, as defined above. Therefore, all UTIs in immunocompromised patients, males, pregnant patients, and those associated with fevers, stones, sepsis, urinary obstruction, catheters, or involving the kidneys are considered complicated infections.

A complicated UTI (cUTI) carries a higher risk of treatment failure compared to a simple UTI. Complicated UTIs involve a broader spectrum of bacteria etiologically and carry a significantly higher risk of clinical complications. Proper identification of a cUTI is crucial as these infections often require longer treatment durations, alternative antibiotics, and additional diagnostic evaluations to ensure effective management. Symptoms of cUTIs include those seen in simple UTIs, such as urinary frequency, urgency, hematuria, dysuria, and suprapubic pain, as well as additional signs like fever, chills, flank pain, sepsis of urological origin, cystitis symptoms persisting for more than seven days, and acute mental status changes (particularly in older adults). Severe cases of cUTIs can escalate to undifferentiated sepsis or septic shock, requiring prompt recognition and management.

Specific examples of a cUTI include: (i) infections despite the presence of anatomical protective measures (UTIs in males are, by definition, considered complicated UTIs); (ii) infections due to anatomical abnormalities, for example, an obstruction, hydronephrosis, renal tract calculi, or colovesical fistula; (iii) infections due to an immunocompromised state, for example, steroid use, postchemotherapy, diabetes, HIV, older individuals; (iv) atypical organisms causing UTI; (v) recurrent infections despite adequate treatment (multidrug-resistant organisms) Infections occurring in pregnancy (including asymptomatic bacteriuria); (vi) infections occurring after instrumentation, such as placing or replacement of nephrostomy tubes, ureteric stents, suprapubic tubes, or Foley catheters; (vii) infections in renal transplant and spinal cord injury patients; (viii) infections in patients with impaired renal function, dialysis, or anuria; and (ix) infections following surgical prostatectomies or radiotherapy.

Most UTIs are due to the colonization of the urogenital tract with rectal and perineal flora. The most common organisms include Escherichia coli, Enterococcus, Klebsiella, Pseudomonas, and other Enterococcus or Staphylococcus species. Of these, E. coli is the most common, followed by Klebsiella. Residential care patients, diabetics, and those with indwelling catheters or immunocompromise can also colonize with Candida. E. coli and possibly Klebsiella overwhelmingly cause simple UTIs. Complicated UTIs tend to be caused by a much wider range of organisms, which is significant because multidrug resistance (MDR) is increasing, and therefore, specific antibiotic treatment regimens vary.

Empiric initial antibiotic selection in septic or systemically ill patients before receiving specific culture results depends on individual patient characteristics and local bacterial resistance antibiograms. Parenteral antibiotics are generally recommended for patients with systemic or severe illness until urine culture results are available to guide the antibiotic selection. Ceftriaxone or piperacillin-tazobactam can be used in patients with less severe illness. Piperacillin-tazobactam is preferred if Enterococcus, Staphylococcus, or Pseudomonas is suspected. Vancomycin, linezolid, or daptomycin should be added if methicillin-resistant Staphylococcus aureus (MRSA) is suspected. If Pseudomonas is suspected, piperacillin-tazobactam, fluoroquinolones, cefepime, or ceftazidime are appropriate. Parenteral fosfomycin has also been used for cUTIs and is effective against many highly resistive organisms, such as ESBL-producing bacteria. Quinolones should be considered when local resistance patterns indicate they are a viable option. Aminoglycosides may be used when other less nephrotoxic drugs cannot be used due to resistance or allergy. For critically ill patients requiring maximum coverage, a combination of a carbapenem with anti-pseudomonal activity, such as imipenem (effective against ESBL-producing organisms), and vancomycin (targeting MRSA), may be appropriate.

The development of MDR organisms has resulted in the reexamination of older antimicrobials (e.g., aminoglycosides and tetracyclines), the development of new antibiotics, and the use of various drug combinations for treating MDR infections (e.g., aztreonam/avibactam; cefepime-enmetazobactam; cefepime-zidebactam; cefiderocol; ceftazidime/avibactam; ceftolozane/tazobactam; eravacycline; pfosfomycin; glycylcyclines; imipenem/relebactam; meropenem/vaborbactam; omadacycline; ivmecillinam; plazomicin; and tebipenem).

Inadequate treatment of cUTIs increases the likelihood of an early recurrence or even an outright failure of therapy. The infection can spread to other organs, result in an abscess, or progress to sepsis and sometimes death. Multidrug-resistant infections are becoming a significant source of in-hospital morbidity and mortality. Suppressive antibiotic regimens are sometimes used in cases where patients respond poorly or are resistant. The FDA recommends using dual primary endpoints to assess the eradication of cUTIs: a clinical response (symptom resolution with no new UTI symptoms) and a microbiological response (urine culture demonstrating <1000 CFU/mL). Long-term antibiotic prophylaxis must be used with caution, as it increases the risk of resistance and changes the susceptibilities of colonized organisms.

Complicated UTIs must be treated more carefully to best serve patients with such infections and avoid the overuse and misuse of antibiotics that ultimately results in more resistant infections. Using the proper antibiotic for an appropriate duration is a critical aspect of antibiotic stewardship, which is defined as the responsible use of antibiotics to ensure effectiveness for future generations by focusing on using the proper drug, at the proper dosage, for the proper duration of treatment, and for the proper indication (i.e., only when truly needed), to combat growing antibiotic resistance, improve patient safety, and reduce side serious effects (e.g., secondary infections).

In the United States, cUTIs account for over 626,000 hospital admissions annually, representing approximately 1.8% of all hospitalizations. UTIs are the seventh most common reason for a patient to be seen in an emergency room in the U.S., constituting over 1 million visits annually. UTIs, particularly complicated ones, are common in hospital settings and often contribute to clinical uncertainty and diagnostic challenges, leading to a significant number of inappropriate antibiotic prescriptions.

According to various publicly available sources, the annual cost of complicated urinary tract infections (cUTIs) in the U.S. is over $6 billion, driven by factors such as inpatient admissions, hospital stays, and repeat treatments. The total cost is substantial because of the high incidence of cUTIs and associated healthcare needs, including repeated doctor visits, emergency room visits, and hospital admissions. About 20% of all bacteremias associated with health care originate from the urinary tract. The mortality associated with these urinary tract-based bacteremias can be up to 10%. About 9.4% of all urological inpatients developed a cUTI during their hospital stay. Regarding the cost per case, the median 30-day healthcare cost for a single cUTI case is approximately $2,000, but this can increase significantly due to complications. Regarding inpatient costs, hospital admissions are the most substantial component of these costs. The median 30-day cost for an inpatient cUTI admission is around $9,441, with a median hospital stay of 4 to 7 days. Regarding outpatient costs, for outpatient cUTIs, the median 30-day cost is approximately $1,531. Recurrent infections and multiple antibiotic treatments are a major driver of these costs. The annual cost for cUTI-related emergency department visits alone was estimated to be $3.2 billion in 2018.

As is apparent from the statistics in the preceding paragraphs, existing standard of care (SOC) methodologies for treating UTIs (although effective in many cases involving non-complicated UTIs) are inadequate and ineffective with regard to cUTIs, which can easily evolve into life-threatening situations. Accordingly, there is an ongoing need for more rapid, more, accurate, more reliable, and more cost-effective systems and methods for diagnosing and treating CUTIs.

### SUMMARY

The following provides a summary of certain example implementations of the disclosed inventive subject matter. This summary is not an extensive overview and is not intended to identify key or critical aspects or elements of the disclosed inventive subject matter or to delineate its scope. However, it is to be understood that the use of indefinite articles in the language used to describe and claim the disclosed inventive subject matter is not intended in any way to limit the described inventive subject matter. Rather the use of “a” or “an” should be interpreted to mean “at least one” or “one or more”.

It should be appreciated that all combinations of the foregoing concepts and additional concepts discussed in greater detail below (provided such concepts are not mutually inconsistent) are contemplated as being part of the inventive subject matter disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the drawings and associated descriptions are to be regarded as illustrative and not restrictive in nature.

A first example embodiment of the disclosed technology provides a direct-from-sample antimicrobial susceptibility testing system, comprising a biological sample taken directly from a patient, wherein the biological sample is processed to capture microorganisms present in the biological sample; an acclimatization medium into which the captured microorganisms are directly transferred to create a test sample, wherein the acclimatization medium is operative to activate protein biosynthesis in the microorganisms in the test sample after a predetermined period of time; a library of antimicrobials at predetermined concentrations to which the test sample is exposed for a predetermined period of time, wherein the exposure of the microorganisms in the test sample to the library of antimicrobials either kills or blocks protein biosynthesis in the microorganisms that are sensitive to one or more of the antimicrobials at one or more of the predetermined concentrations; a non-canonical amino acid, wherein the non-canonical amino acid is incorporated into newly biosynthesized proteins in the microorganisms that initially survive exposure to the antimicrobials thereby labeling the newly biosynthesized proteins; and a detectable tag configured to attach to the non-canonical amino acid labels in the newly biosynthesized proteins, wherein tagging the labeled proteins with the detectable tag creates an amount of detectable signal.

Implementations of the first example embodiment further comprise a system or device for detecting the signal. Certain implementations further comprise a system or device for comparing the amount of detected signal in the test sample to a positive control, wherein the positive control is a test sample that has been labeled and tagged but that has not been exposed to the library of antimicrobials, wherein an observed absence of or a decrease in the amount of detectable signal relative to the positive control indicates susceptibility of the living microorganisms to one or more of the antimicrobials in the library of antimicrobials at one or more of the predetermined concentrations, and wherein an observed signal that approaches or is equal to the value of the positive control indicates resistance of the living microorganisms to one or more of the antimicrobials in the library of antimicrobials at one or more of the predetermined concentrations.

In certain implementations of the first example embodiment, the patient is suspected of having an infection, such as a complicated urinary tract infection, caused by at least one pathogenic microorganism that is suspected of being present in the biological sample. In certain implementations, the biological sample is derived from a bodily fluid or other bodily source, which may include urine. In various implementations, the microorganisms include bacteria, the library of antimicrobials includes antibiotics, and the non-canonical amino acid is an alkyne-modified non-canonical amino acid, which may be L-Homopropargylglycine (HPG). In certain implementations, the newly biosynthesized proteins include surface proteins, internal proteins, or a combination of surface proteins and internal proteins of the microorganisms in the test sample. In certain implementations, the detectable tag is an azide-modified detection molecule that is reacted with the alkyne-modified non-canonical amino acid using click-chemistry and the azide-modified detection molecule is a biotinylated ligand, a fluorogenic or fluorescent azide probe, or a fluorogenic or fluorescent dye. In certain implementations, the detectable signal is generated using a method that is fluorescence-based, enzyme-linked immunosorbent assay (ELISA)-based, cell-based, dot blot-based, or microscopy-based. In certain implementations, the signal is quantifiable, and a predetermined amount of detected signal is indicative of a minimum inhibitory concentration (MIC) of antibiotic. In certain implementations, the system is a high-throughput method executed on a multi-well plate or microplate, wherein the type of multi-well plate or microplate includes filtration plates, and wherein more than one type of antimicrobial may be tested on the multi-well plate or microplate.

A second example embodiment of the disclosed technology provides a direct-from-sample method for determining antimicrobial susceptibility, comprising obtaining a biological sample taken directly from a patient; processing the biological sample to capture microorganisms present in the biological sample; transferring the captured microorganisms directly into an acclimatization medium to create a test sample, wherein after a predetermined period of time, the acclimatization medium activates protein biosynthesis in the microorganisms in the test sample; exposing the test sample to a library of antimicrobials at predetermined concentrations for a predetermined period of time, wherein the exposure of the microorganisms in the test sample to the library of antimicrobials either kills or blocks protein biosynthesis in the microorganisms that are sensitive to one or more of the antimicrobials at one or more of the predetermined concentrations; labeling newly synthesized proteins in the microorganisms that initially survive exposure to the antimicrobials using a non-canonical amino acid that incorporates into the newly biosynthesized proteins; attaching a detectable tag to the non-canonical amino acids incorporated into the newly biosynthesized proteins, wherein tagging the labeled proteins with the detectable tag creates an amount of detectable signal; and detecting the amount of signal present in the test sample.

Implementations of the second example embodiment further comprise comparing the amount of detected signal in the test sample to a positive control, wherein the positive control is a test sample that has been labeled and tagged but that has not been exposed to the library of antimicrobials, wherein an observed absence of or a decrease in the amount of detectable signal relative to the positive control indicates susceptibility of the living microorganisms to one or more of the antimicrobials in the library of antimicrobials at one or more of the predetermined concentrations, and wherein an observed signal that approaches or is equal to the value of the positive control indicates resistance of the living microorganisms to one or more of the antimicrobials in the library of antimicrobials at one or more of the predetermined concentrations.

In certain implementations of the second example embodiment, the patient is suspected of having an infection, such as complicated urinary tract infection, caused by at least one pathogenic microorganism that is suspected of being present in the biological sample. In certain implementations, the biological sample is derived from a bodily fluid or other bodily source, which may include urine. In various implementations, the microorganisms include bacteria, the library of antimicrobials includes antibiotics, and the non-canonical amino acid is an alkyne-modified non-canonical amino acid, which may be L-Homopropargylglycine (HPG). In certain implementations, the newly biosynthesized proteins include surface proteins, internal proteins, or a combination of surface proteins and internal proteins of the microorganisms in the test sample. In certain implementations, the detectable tag is an azide-modified detection molecule that is reacted with the alkyne-modified non-canonical amino acid using click-chemistry and the azide-modified detection molecule is a biotinylated ligand, a fluorogenic or fluorescent azide probe, or a fluorogenic or fluorescent dye. In certain implementations, the detectable signal is generated using a method that is fluorescence-based, enzyme-linked immunosorbent assay (ELISA)-based, cell-based, dot blot-based, or microscopy-based. In certain implementations, the signal is quantifiable, and a predetermined amount of detected signal is indicative of a minimum inhibitory concentration (MIC) of antibiotic. In certain implementations, the system is a high-throughput method executed on a multi-well plate or microplate, wherein the type of multi-well plate or microplate includes filtration plates, and wherein more than one type of antimicrobial may be tested on the multi-well plate or microplate.

A third example embodiment of the disclosed technology provides a direct-from-sample method for determining antimicrobial susceptibility, comprising obtaining a biological sample taken directly from a patient; processing the biological sample to capture microorganisms present in the biological sample; transferring the captured microorganisms directly into an acclimatization medium to create a test sample, wherein after a predetermined period of time, the acclimatization medium activates protein biosynthesis in the microorganisms in the test sample; exposing the test sample to a library of antimicrobials at predetermined concentrations for a predetermined period of time, wherein the exposure of the microorganisms in the test sample to the library of antimicrobials either kills or blocks protein biosynthesis in the microorganisms that are sensitive to one or more of the antimicrobials at one or more of the predetermined concentrations; labeling newly synthesized proteins in the microorganisms that initially survive exposure to the antimicrobials using a non-canonical amino acid that incorporates into the newly biosynthesized proteins; attaching a detectable tag to the non-canonical amino acids incorporated into the newly biosynthesized proteins, wherein tagging the labeled proteins with the detectable tag creates an amount of detectable signal; detecting the amount of signal present in the test sample; comparing the amount of detected signal in the test sample to a positive control, wherein the positive control is a test sample that has been labeled and tagged, but has not been exposed to the library of antimicrobials, wherein an observed absence of or a decrease in the amount of detectable signal relative to the positive control indicates susceptibility of the living microorganisms to one or more of the antimicrobials in the library of antimicrobials at one or more of the predetermined concentrations, and wherein an observed signal that approaches or is equal to the value of the positive control indicates resistance of the living microorganisms to one or more of the antimicrobials in the library of antimicrobials at one or more of the predetermined concentrations.

In certain implementations of the third example embodiment, the patient is suspected of having an infection, such as a complicated urinary tract infection, caused by at least one pathogenic microorganism that is suspected of being present in the biological sample. In certain implementations, the biological sample is derived from a bodily fluid or other bodily source, which may include urine. In various implementations, the microorganisms include bacteria, the library of antimicrobials includes antibiotics, and the non-canonical amino acid is an alkyne-modified non-canonical amino acid, which may be L-Homopropargylglycine (HPG). In certain implementations, the newly biosynthesized proteins include surface proteins, internal proteins, or a combination of surface proteins and internal proteins of the microorganisms in the test sample. In certain implementations, the detectable tag is an azide-modified detection molecule that is reacted with the alkyne-modified non-canonical amino acid using click-chemistry and the azide-modified detection molecule is a biotinylated ligand, a fluorogenic or fluorescent azide probe, or a fluorogenic or fluorescent dye. In certain implementations, the detectable signal is generated using a method that is fluorescence-based, enzyme-linked immunosorbent assay (ELISA)-based, cell-based, dot blot-based, or microscopy-based. In certain implementations, the signal is quantifiable, and a predetermined amount of detected signal is indicative of a minimum inhibitory concentration (MIC) of antibiotic. In certain implementations, the system is a high-throughput method executed on a multi-well plate or microplate, wherein the type of multi-well plate or microplate includes filtration plates, and wherein more than one type of antimicrobial may be tested on the multi-well plate or microplate.

### BRIEF DESCRIPTION OF THE DRAWINGS

The accompanying drawings and figures, which are incorporated into and form a part of the specification, schematically illustrate one or more example implementations of the disclosed inventive subject matter and, together with the general description given above and detailed description given below, explain the principles of the disclosed subject matter.

FIG. 1 is graphic representation of the general workflow of the BLAST assay;

FIG. 2 depicts a click chemistry reaction scheme using Cu (I)-catalyzed azide-alkyne cycloaddition (CuAAC);

FIG. 3A depicts the process of labeling a bacterial protein with an alkyne-modified non-canonical amino acid and reacting the alkyne-modified non-canonical amino acid with an azide-modified detection molecule using click-chemistry.

FIG. 3B depicts the BLAST bacterial labeling and tagging process using click-chemistry. The bacterial sample is acclimatized, treated with antibiotics and newly synthesized proteins in metabolically viable cells are labeled with HPG. Labeled proteins are tagged with a fluorescent dye using click-chemistry.

FIG. 4 is a flowchart depicting an example stepwise process for completing the BLAST assay.

FIG. 5 is a graphic comparing the stepwise process of FIG. 4 to a prior art clinical AST workflow;

FIG. 6 is a graphic illustration of an example BLAST assay filtration plate layout. In this implementation, all control wells (column 1) will not receive antibiotics, but will receive a tagging buffer (Buffer C1/C2 mix). Sterility control wells (column 1, rows A and B) will not receive bacteria, but will receive a labeling buffer (Buffer B). Positive control wells (column 1, rows C-F) will receive bacteria, a labeling buffer (Buffer B) and a tagging buffer (Buffer C1/C2 mix), but will not receive antibiotics. Background control wells (column 1, rows G and H) will receive bacteria and tagging buffer (Buffer C1/C2 mix), but will not receive labeling buffer (Buffer B) and antibiotics.

FIGS. 7A-7B are bar charts depicting the results of a study performed to evaluate the BLAST labeling response as a function of bacterial concentration in the absence of antibiotics using E. coli ATCC 25922 and S. aureus ATCC 29213. The BLAST assay was performed using isolates with varying inoculum ranging from 1.5×10¹ CFU/mL to 1.5×10⁸ CFU/mL. In each assay the positive control bacterial samples were treated with the labeling buffer and the tagging buffer mix while the background control bacterial samples were not treated with the labeling buffer but were treated with the tagging buffer. Experiments were repeated in triplicate and error bars represent the standard deviation. The labeling response was assessed by comparing fluorescence signals from test samples to those of background controls, enabling evaluation of signal strength across the inocula range. FIG. 7A depicts the results for E. coli ATCC 25922, and FIG. 7B depicts the results for S. aureus ATCC 29213.

FIGS. 8A-8C are bar charts depicting the use of the BLAST assay to determine MIC values for E. coli ATCC 25922 and S. aureus ATCC 29213 against nitrofurantoin at a starting inoculum of 5.0×10⁶ CFU/mL. Eight concentrations of nitrofurantoin were tested including the CLSI susceptibility breakpoint, four concentrations below the breakpoint and three concentrations above the breakpoint in two-fold dilution. FIG. 8A depicts the BLAST fluorescent signal for E. coli ATCC 25922 against nitrofurantoin. FIG. 8B depicts the BLAST fluorescent signal for S. aureus ATCC 29213 against nitrofurantoin. In both panels, the experiments were repeated in triplicate and error bars represent the standard deviation. FIG. 8C depicts the Relative Response Ratio (RRR) shows the relative labeling efficiency of the bacteria. The RRR was determined based on the formula described in the methods and the BLAST MIC is the lowest antibiotic concentration at which the RRR equals or surpasses the BLAST cutoff point (0.8). In this case, the BLAST MIC was 8 μg/mL for both strains, which agrees with the CLSI QC range of 4-16 μg/mL and 8-32 μg/mL for E. coli ATCC 25922 and S. aureus ATCC 29213 respectively.

### DETAILED DESCRIPTION

Example implementations are now described with reference to the Figures. Reference numerals are used throughout the detailed description to refer to the various elements and structures. Although the following detailed description contains many specifics for the purposes of illustration, a person of ordinary skill in the art will appreciate that many variations and alterations to the following details are within the scope of the disclosed inventive subject matter. Accordingly, the following implementations are set forth without any loss of generality to, and without imposing limitations upon, the claimed subject matter. The terms “antibiotic” and “antimicrobial” are used interchangeably throughout this disclosure, as are the terms “bacteria” and “microorganisms”, and the use of one term should not be construed as limiting the scope, meaning, or definition of the other term in any way.

As previously discussed, conventional antimicrobial susceptibility testing (AST) requires culture, isolation, and prolonged incubation to identify bacterial growth inhibition in response to tested antibiotics. The disclosed technology utilizes metabolic labeling of active bacteria using non-canonical amino acid incorporation rather than culture and isolation which is required by more traditional AST methods. BLAST is a phenotypic assay that determines antibiotic susceptibility by monitoring bacterial viability and metabolic activity following exposure to various preselected antibiotics. This is accomplished through a series of steps involving the incubation of bacteria within a range of antibiotic concentrations, followed by labeling (e.g., using fluorescence) and detection of the labeled bacteria. This method measures the bacterial response to antibiotics without relying on any ability of the bacteria to replicate or form concentrated cultures such as those used in routine urine culture, which is described in detail below for the purpose of providing background and context for the description of BLAST, which provides significant advantages over routine urine culture.

### Routine Urine Culture

Routine urine culture, which is the current gold standard for diagnosing a UTI, is typically performed when a physician suspects an infection based on symptoms such as painful urination and/or an unexplained fever, or to check for asymptomatic bacteriuria in pregnant women. Routine urine culture is used to: (i) confirm the presence of a UTI; (ii) identify the cause of the UTI by determining the type of bacteria or yeast causing the infection; and (iii) determine an effective treatment for the UTI (e.g., sensitivity testing to determine which antibiotics will be effective against the identified microorganism). Urine culture may also be performed after treatment with an antibiotic to confirm that an infection has been successfully treated. Routine urine culture typically involves the sequential microbiological steps of: (i) culture; (ii) quantitation; (iii) isolation; and (iv) identification. These steps are often used together in a diagnostic workflow to detect infections, identify the causative pathogen, and determine appropriate treatment, such as determining to which antibiotic an identified pathogen is susceptible and at what concentration.

Culture of Microorganisms from Urine Sample

Culture involves growing microorganisms in a controlled environment, such as in a petri dish containing a nutrient-rich substance (agar) or in another type of growth medium. Culture can take 24-48 hours or longer to achieve a large enough population of microorganisms for analysis. The culture step can be qualitative, wherein the presence or the absence of a specific organism is determined; or quantitative/semi-quantitative, wherein the number of organisms present is determined.

### Quantitation of Microorganisms in Urine Sample

Quantitation involves determining the number of microorganisms present in a sample, typically reported as colony-forming units per milliliter (CFU/mL). A urine culture might detect bacteria at levels above 10⁴ CFU/mL. Quantitation is particularly important for clinical samples such as urine for differentiating between normal colonization/contamination and a true infection that requires treatment. Both direct and indirect methods may be used to quantify microbial growth and measure either the total number of cells or only the number of viable (live) cells. Direct methods of quantitation involve directly counting cells or colonies and include: (i) plate counts (CFU); (ii) direct microscopic count; (iii) membrane filtration; and (iv) most probable number (MPN). counted under a microscope. Indirect methods of quantitation measure physical or chemical parameters that change with cell number and include: (i) turbidity measurement (optical density); (ii) dry weight; and (iii) measurement of metabolic activity. The choice of method depends on the specific requirements of the test, such as required accuracy, time available, and whether the number of live or total cells is required.

Isolation of Pathogen from Culture

Isolation is the technique of separating a single, pure strain of microorganism from a mixed population. Isolation is often achieved by streaking a culture sample onto an agar plate and allowing individual cells to grow into distinct colonies that can then be sub-cultured. The purpose of isolation is to ensure that subsequent identification and susceptibility testing are performed on a single species, not a mixture of different organisms. Typically, a culture sample is streaked across an agar plate to obtain individual, well-separated colonies. Each distinct colony is presumed to be a pure culture derived from a single cell.

### Identification of Pathogen

Identification is the precise characterization of an isolated microorganism to determine its species or type. The purpose of identification is to determine the specific pathogen causing an infection and guide appropriate treatment, such as selecting an appropriate antibiotic. Methods for identification include phenotypic, genotypic, and proteotypic identification. Phenotypic identification involves observing characteristics such as colony morphology, Gram stain reaction, and biochemical testing (e.g., using commercial kits (e.g., API systems) or automated systems that utilize biochemical profiles (e.g., VITEK® 2 system). Genotypic identification uses molecular techniques such as polymerase chain reaction (PCR) or DNA sequencing to analyze the genetic material of an organism. Proteotypic identification uses mass spectrometry (e.g., MALDI-TOF MS) to analyze protein profiles. Automated biochemical testing, molecular testing, and mass spectrometry typically produce rapid results compared to slower, more traditional phenotypic methods of identification.

### Determining Susceptibility of Pathogen to Antimicrobials

Antimicrobial Susceptibility Testing (AST) is a laboratory procedure that identifies which antibiotics (or other antimicrobials) will effectively kill or stop the growth of a specific bacteria or fungus (or other microorganism) causing an infection, thereby guiding physicians to the most effective treatment while combating rising antimicrobial resistance. Commonly used methods of determining the susceptibility of identified microorganisms to antimicrobials include: (i) disk diffusion; and (ii) broth microdilution.

### Disk Diffusion

Disk diffusion is a laboratory method used for determining the susceptibility or resistance of bacteria to different antibiotics. Disk diffusion involves the following basic steps: (i) a broth suspension of bacteria is uniformly spread across an agar plate, creating a bacterial lawn; (ii) discs impregnated with specific antibiotics are placed on the surface of the agar in the plate; (iii) the plate is incubated, and the microorganisms grow; (iv) the antibiotic diffuses from the disc into the agar; (v) if the bacteria growing on the plate are susceptible to the antibiotic, their growth is inhibited around the disc, creating a clear area called the “zone of inhibition”; and (vi) the diameter of this zone is measured and compared to standard interpretive guidelines to determine if the bacteria are susceptible, intermediate, or resistant to the antibiotic. Disk diffusion has applications in antimicrobial susceptibility testing (AST), which is widely used to test bacteria against multiple antibiotics simultaneously and guide the selection of an effective treatment, and in epidemiology for identifying similarities and differences between strains and determining if a single strain is likely causing multiple infections.

### Broth Microdilution

Broth microdilution (BMD) is used to determine the antibiotic susceptibility of a bacteria by exposing the bacteria to a series of two-fold dilutions of an antibiotic in a liquid growth medium. BMD identifies the minimum inhibitory concentration (MIC), which is the lowest concentration of the antibiotic that prevents visible microbial growth. BMD involves the following basic steps: (i) a range of two-fold dilutions of an antibiotic are prepared in a liquid growth medium, typically in the wells of a 96-well plate; (ii) a standardized number of microorganisms (inoculum) are added to each well; (iii) the plates are incubated for a set period, usually 12-24 hours at a specific temperature (e.g., 37° C.); and (iv) the MIC is identified as the concentration of the antibiotic in the first well where there is no visible or detectable bacterial growth after incubation. Currently, BMD is the most used method for determining MIC values and is considered the gold standard for accuracy.

### Antibiotic Breakpoints

Antibiotic breakpoints are crucial concentration thresholds used by laboratories to interpret AST results by setting specific antibiotic concentrations that distinguish susceptible organisms (i.e., likely to respond to treatment) from resistant ones (i.e., unlikely to respond to treatment) for guiding effective treatment decisions. The critical values for these concentration thresholds are set by organizations such as Clinical and Laboratory Standards Institute (CLSI), United States Food and Drug Administration (FDA), and the European Committee on Antimicrobial Susceptibility Testing (EUCAST) based on drug data, bacterial characteristics, and patient outcomes. Based on AST results, microorganisms are categorized as “Susceptible(S)”; “Susceptible-Dose-Dependent (SDD)”; “Intermediate (I)”; or “Resistant (R)”. “Susceptible” organisms are those for which treatment with standard doses of antibiotic is likely to be successful. “Susceptible-Dose-Dependent” organisms are those for which higher or more frequent doses of antibiotic may be required for successful treatment. “Intermediate” organisms are those for which an antibiotic may work at maximum does, but successful treatment is uncertain. “Resistant” organisms are those that are not likely to respond to a particular antibiotic. A laboratory determines the MIC (i.e., lowest concentration to inhibit bacterial growth) and then compares the MIC to the breakpoint value to arrive at a final S/I/R result. A bacterial MIC below the breakpoint indicates that a particular antibiotic will be effective, thereby guiding doctors to choose drugs that successfully treat infections.

### BLAST™

As disclosed in U.S. patent application Ser. Nos. 17/965,207 and 18/907,908, both of which are incorporated by reference herein, in their entirety for all purposes, the BLAST™ (Bacteria Labeling Antibiotic Susceptibility Test) platform (hereinafter referred to simply as BLAST), which was developed for use with UTIs and cUTIs in particular, utilizes metabolic labeling of active (i.e., living) bacteria or other microorganisms using non-canonical amino acid incorporation into newly synthesized internal and external proteins produced by bacteria or other microorganisms present in a biological sample. The newly synthesized proteins are detected by tagging metabolically viable cells with a signal-generating fluorescent dye, labeling only proteins translated after pathogen exposure to antibiotics. The disclosed assay exploits the fact that living bacteria have a significantly faster metabolism and protein production than dead or dying bacteria and will take up amino acids and incorporate them into newly formed proteins at a much faster rate. Because the assay can be performed directly on a biological sample, the culture, isolation, and pathogen identification steps described above for typical routine urine culture are unnecessary. Accordingly, BLAST provides rapid and accurate AST results not achievable using routine urine culture methods.

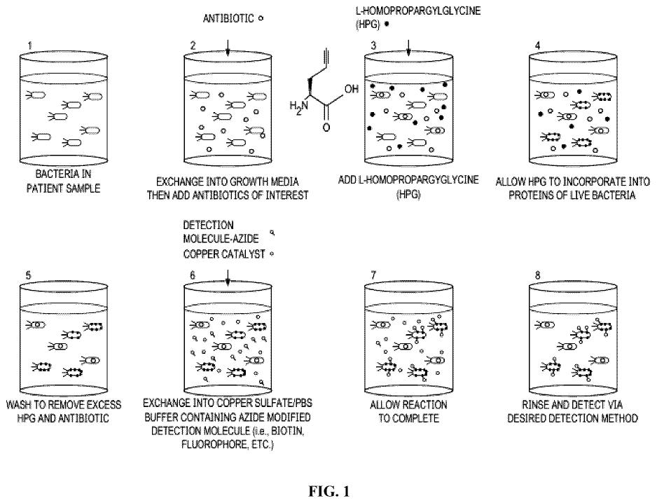

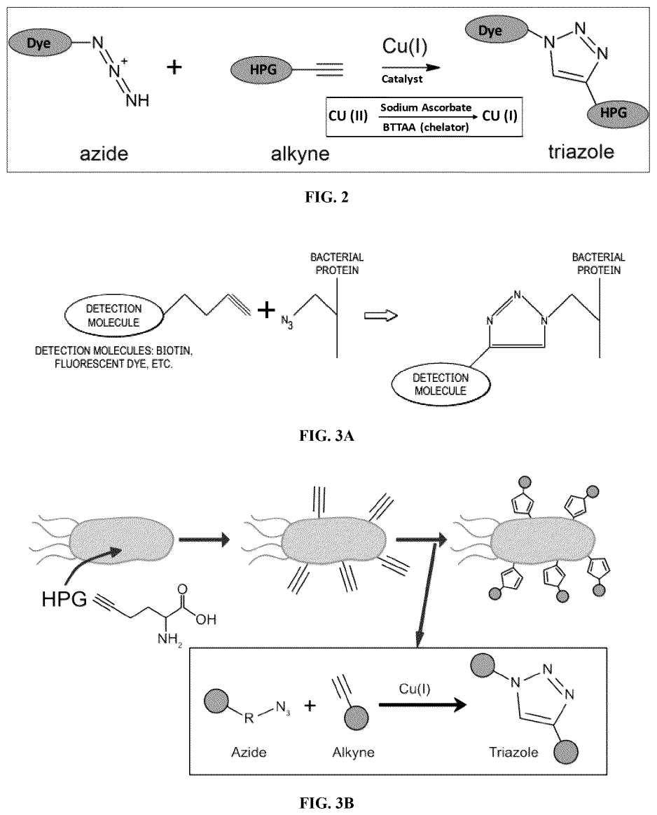

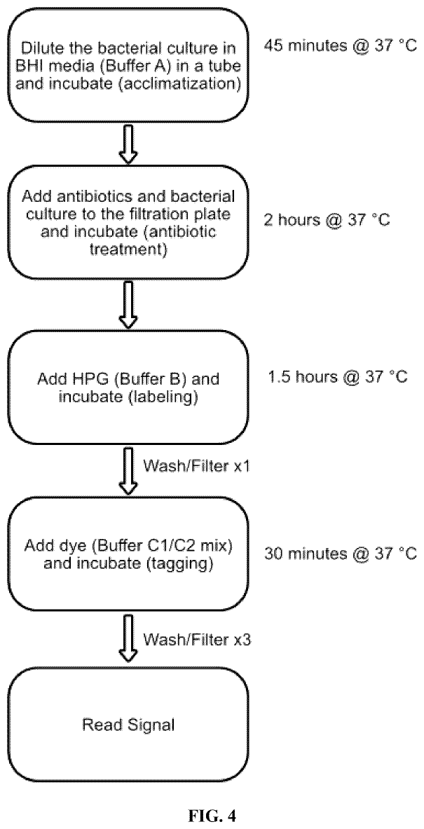

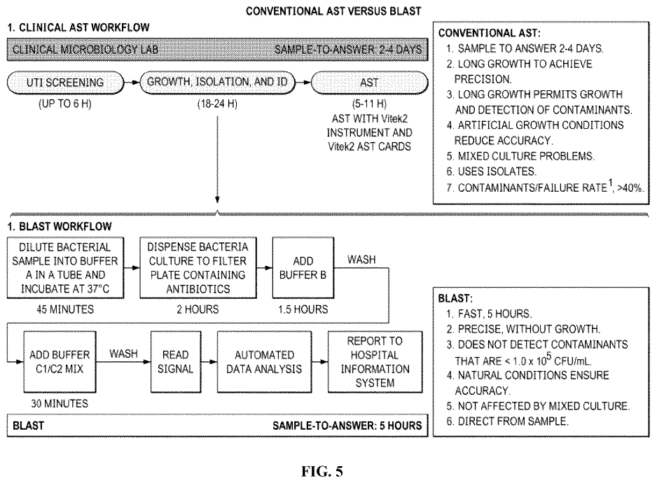

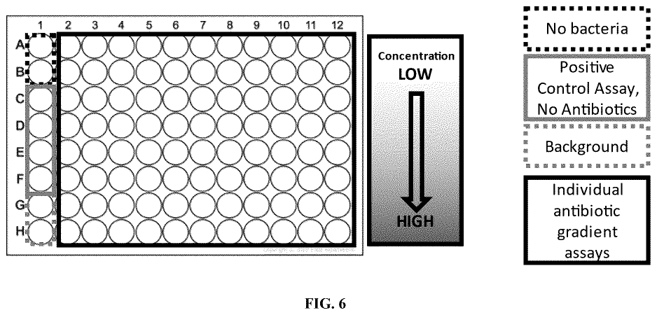

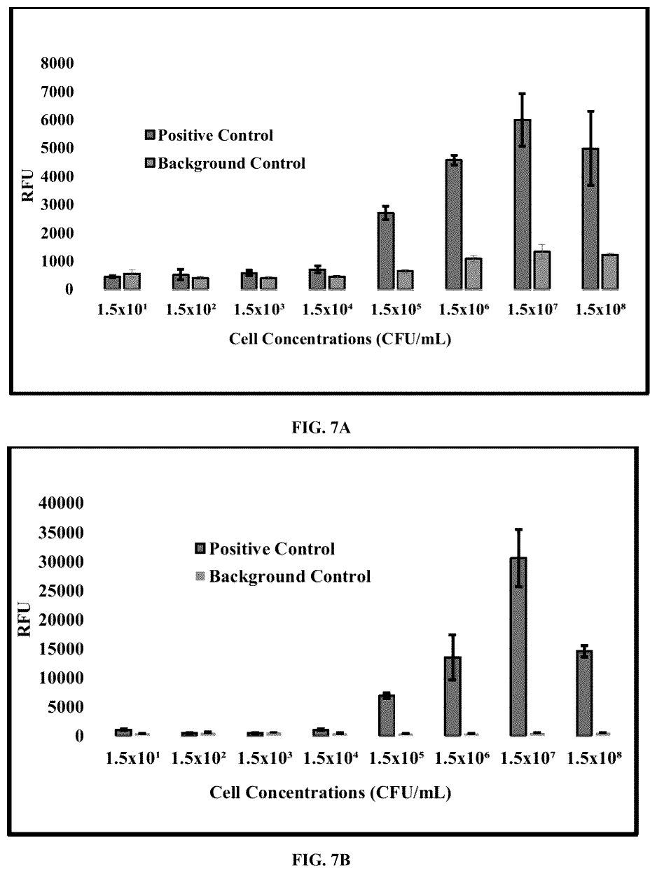

With reference to FIGS. 1, 2, and 3A-3B, implementations of the disclosed assay replace methionine in a bacterial growth media with a non-canonical amino acid (ncAA), which includes a specific reactive group, thereby enabling specific detection of living bacteria (see, for example, Sherratt et al. Rapid Screening and Identification of Living Pathogenic Organisms via Optimized Bioorthogonal Non-canonical Amino Acid Tagging, Cell Chemical Biology 24, 1048-1055 (2017), which is incorporated by reference herein in its entirety). L-Homopropargylglycine (HPG) is an example of an alkyne modified ncAA that mimics methionine during protein production. When HPG is present in bacterial growth media, bacteria growing in the media will incorporate HPG into newly synthesized proteins. Alkyne groups are not naturally found in bacterial cells and serve as a specific reactive group for bacteria undergoing active protein synthesis. HPG has been detected in bacteria after just a 30-minute incubation period, making the entire process comparatively fast. The alkyne group is one component of the copper catalyzed alkyne-azide cycloaddition (CuAAC), more commonly known as click chemistry (see, for example, Atwal et al., Clickable methionine as a universal probe for labelling intracellular bacteria, Journal of Microbiological Methods 169 (2020) 105182; and Li et al., Fluorogenic “click” reaction for labeling and detection of DNA in proliferating cells, BioTechniques 49:525-527 (July 2010), both of which are incorporated by reference herein in their entirety for all purposes). When a ligand with an azide group encounters an alkyne group, the reaction creates an irreversible ring structure (see FIG. 2). After bacterial uptake of HPG, alkyne groups can be found in any protein that includes a methionine amino acid, including internal proteins and surface proteins (FIGS. 3A-3B). Because the CuAAC reaction specifically labels living bacteria at their surface, in some implementations, cell lysis may be eliminated as a necessary aspect of the assay. However, in other implementations, cell lysis may be employed for assay optimization and for increasing the amount of signal generated by the assay. A general workflow for the disclosed assay is shown in FIG. 4. The assay has been demonstrated to detect antibiotic susceptibility in as few as 5-6 hours, depending on patient sample. FIG. 5 provides a graphic comparison of the traditional AST workflow and the BLAST workflow. FIG. 6 is a graphic illustration of an example BLAST assay filtration plate layout.

Some implementations of the disclosed assay utilize variations of click chemistry that do not involve copper catalysis such as, for example, the use of a strained azide or strained alkyne that is highly reactive and does not require catalysis. See, for example, Friscourt et al., A Fluorogenic Probe for the Catalyst-Free Detection of Azide-Tagged Molecules, J Am Chem Soc. 134 (45): 18809-18815 (Nov. 14, 2012), which is incorporated by reference herein, in its entirety, for all purposes. Multiple detection methods can be used with the disclosed assay, including fluorescence, cells, blotting, and ELISA-based methods depending on which detection molecule is chosen. To detect antibiotic susceptibility, the signal produced from a control sample (no antibiotic treatment) is compared with samples treated with antibiotics, thereby detecting changes in bacterial protein production that correlate to antibiotic susceptibility. Complex image-based methods are enabled by using image analysis techniques and artificial intelligence (AI)-based software for determining results.

As disclosed in U.S. patent application Ser. No. 18/907,908, an example embodiment of the BLAST technology provides a test method for rapidly determining the susceptibility of microorganisms (e.g., bacteria) to various antimicrobials (e.g., antibiotics) that includes placing living microorganisms obtained from a native biological into an acclimatization buffer that activates the metabolism of the living microorganisms and initiates protein biosynthesis in those microorganisms. The microorganisms are then exposed to a library of antimicrobials that includes various different antimicrobials at predetermined concentrations. This exposure either kills the microorganisms or blocks protein biosynthesis in the microorganisms that are sensitive to one or more of the antimicrobials at one or more of the predetermined concentrations. Newly biosynthesized proteins produced by the living microorganisms that survive exposure to the antimicrobials are then labeled by incorporating a non-canonical amino acid (ncAA) into the biosynthesized protein (e.g., the ncAA is homopropargylglycine (HPG) which includes an alkyne moiety and the newly biosynthesized proteins, therefore, include the alkyne moiety). The labeled proteins are then tagged with a detectable element by attaching the detectable element to the non-canonical amino acid (e.g., the detectable element is a fluorophore-tagged dye that includes an azide group that reacts with the alkyne moiety of HPG). Tagging the labeled proteins with the detectable element creates an amount of detectable signal which is detected and compared to a positive control. An observed absence of or a decrease in the amount of detectable signal relative to the positive control indicates effectiveness of one or more of the antimicrobials in the library of antimicrobials against the living microorganisms at one or more of the predetermined concentrations. An observed signal that approaches or is equal to the value of the positive control indicates ineffectiveness of one or more of the antimicrobials in the library of antimicrobials against the living microorganisms at one or more of the predetermined concentrations. Certain implementations of the BLAST test method further comprise using the absence of or decrease in detectable signal at a particular concentration of an effective antimicrobial to determine a minimum inhibitory concentration (MIC) for each effective antimicrobial in the library of antimicrobials.

### Blast Comparative Study and Results

The description of the BLAST assay appearing below is based on the following publication: A Rapid Bacterial Labeling Method for Phenotypic Antimicrobial Susceptibility Testing (Diagnostic Microbiology and Infectious Disease. Volume 114, Issue 2, February 2026, 117181), which is incorporated by reference herein, in its entirety, for all purposes. This publication discloses a scientific study that supports direct-from-sample testing using BLAST.

As previously discussed herein, bacterial infections pose significant treatment challenges due to the increasing prevalence of multidrug-resistant pathogens. Current Antimicrobial Susceptibility Test (AST) workflows, including automated systems, require culture, isolation, and identification steps, which are time-consuming and may negatively affect accuracy. Providing timely and more accurate AST results can lead to better patient outcomes, more effective antibiotic stewardship, and significant cost savings. The study described below evaluated BLAST as a diagnostic method, using a selection of strains typically found in urinary tract infections and antibiotics known to inhibit protein biosynthesis. The BLAST assay provided accurate AST results within 5-6 hours, as demonstrated using reference pathogens grown in broth, agar plates, or as bacteria-contrived urine samples.

As previously discussed herein, example embodiments of the BLAST assay use click-chemistry to tag newly synthesized proteins in common bacteria and measure signal inhibition by frequently prescribed antibiotics (e.g., agents that target cell wall biosynthesis and nucleic acid replication). The study described below was conducted over a concentration range (10⁵ to >10⁸ CFU/mL) for two reference strains. Minimum inhibitory concentrations (MICs) were determined for 11 antibiotics across multiple Gram-positive and Gram-negative pathogens using a fluorescent reader in a 96-well filtration plate format. Test results were compared to reference broth microdilution (rBMD) values. The assay was shown to detect the inhibition of new protein biosynthesis using disparate classes of antibiotics that block cell wall formation or macromolecular synthesis, with MIC values comparable to rBMD and expected results for CLSI reference strains. Testing of bacteria-contrived urine cultures also demonstrated good agreement with results from isolated colonies tested with either BLAST or rBMD. Accordingly, BLAST offers a unique pathway to a rapid, scalable, and more accurate solution for AST.

### Introduction

As previously discussed herein, UTIs are among the most common causes of hospital admissions and are a significant contributor to antibiotic consumption in hospitals and general healthcare. Typically, a cUTI is accompanied by symptoms which suggest an infection extending beyond the bladder [1], [2], [3]. In the United States, there are over 626,000 hospital admissions annually for cUTIs, comprising about 1.8% of all hospitalizations [4]. Simple UTIs can often be managed with outpatient antibiotics, typically resulting in favorable outcomes. However, cUTIs pose a greater risk as they can lead to severe urosepsis, which can be fatal.

Detection, identification, and antimicrobial susceptibility testing of pathogens using traditional methods can be lengthy because they rely on selective culturing and biochemical assays [5]. Conversely, automated systems using turbidity or dye accumulation have improved efficiency and can be accomplished in 6 to 12 hours, but still require prior bacterial isolation or culturing, making it challenging to perform the test in <24 hours [6]. Rapid and highly specific methods of pathogen assessment have been developed including nucleic acid-based methods and immunological assays which can significantly speed up the pathogen detection process [7,5]. However, unlike metabolic labeling methods that can reveal pathogen viability, nucleic acid-based methods and immunological assays do not determine whether the detected target analyte originated from viable metabolically active cells. Development of systems that can result in shorter sample-to-result time would allow the practitioner to prescribe a more targeted therapy or switch to the more effective treatment earlier, and is anticipated to improve patient outcomes, lower costs, and support more effective antibiotic stewardship.

Functional prototypes of rapid, direct-from-sample tests have indicated some practical pathways to improved diagnostics. A resazurin-based assay [8] sensitive enough to detect the response of individual bacteria, to antibiotics in a shortened time frame has been described. However, this method was demonstrated using only E. coli and a limited number of antimicrobial agents. An alternative single-cell impedance cytometric AST method, iFAST [9] relies on electrical impedance to monitor bacterial activity following antimicrobial exposure. Other methods that use specific enzymes such as beta glucuronidase indicate that AST results can be obtained by monitoring bacterial metabolic activity [10], [11], [12]] and feature the use of filtration during the assay. The BLAST method described herein builds on that observation with the goal of making a practical assay that does not require specialized cartridge manufacture and is focused on scalability and generating MIC values for a range of clinically important antibiotics.

BLAST provides a unique approach that significantly reduces the time for AST testing to 5-6 hours (FIG. 5). BLAST is a metabolic labeling method [13,14] that detects newly synthesized proteins by labeling only proteins translated after pathogen exposure to antibiotics using the non-canonical amino acid, homopropargylglycine (HPG) (FIG. 3B). The labeled, metabolically viable cells are then tagged with the fluorescent dye AZDye 488 Azide Plus (Vector Laboratories, Cat. #CCT-1475). The assay detects proteins and does not require extensive sample processing. This method, adapted from bioorthogonal labeling techniques [15,16] used in microscopy, is evaluated here as a means of determining antibiotic susceptibility using a variety of bacteria and drug combinations. The test is designed to rapidly determine the antibiotic susceptibility of bacterial strains, assessing their resistance or sensitivity to various antibiotics.

### Materials and Reagents

Bacterial strains were obtained from ATCC (E. coli ATCC 25922, S. aureus ATCC 29213, and K. pneumoniae ATCC 700603) and NCTC (A. baumannii NCTC 13304). Another isolate (E. coli LSI 770) was obtained from Laboratory Specialists, Inc., Westlake, OH. E. coli LSI 770 is a multiple-drug-resistant isolate that was used in the assays to compare with the susceptible strains. Sterile pooled human urine samples were obtained from Innovative Research, Novi MI, and were batch-tested to rule out bacterial contamination. The BLAST assay was performed on a 0.2 μm 96-well Supor (Polyethersulfone) filtration plate purchased from Cytiva (Cat. #8019). All isolates were cultured overnight on trypticase soy agar with 5% of sheep's blood (VWR, 90001-282) prior to testing. To perform the BLAST assay, bacterial samples were acclimatized in Brain Heart Infusion (BHI) media obtained from Sigma (Cat. #53286), labeled with 1-Homopropargylglycine (HPG) purchased from Vector Laboratories (Cat. #CCT1067) and tagged with AZDye 488 Azide Plus dye from Vector Laboratories (Cat. #CCT-1475). The tagging process was accomplished by copper catalyzed azide alkyne cycloaddition (Click-Chemistry) [16], [17], [18]] using BTTAA (Vector Laboratories, Cat. #CCT-1236) as a catalyst and sodium ascorbate (Sigma, Cat. #A7631) as a reducing agent. The antibiotics used in this study (Table 1) were procured from either Sigma or MedChem Express. BLAST buffers (Table 2) were made in the laboratory or provided by DCN Diagnostics, Carlsbad, CA. Antibiotic stocks were prepared using DMSO or water (200-fold higher than the highest test concentrations) and stored at −20° C. A plate seal (VWR Cat. #60941-064) and AeraSeal™ plate adhesive (VWR Cat. #490007-322) were used for covering the bottom and the top of the filtration plate, respectively during the incubation steps in the assay when the plate was not being filtered. Plates were filtered and washed using the vacuum manifold and pump system from Sterlitech (Cat #195200-11 and 167900-11).

TABLE 1
The list of antibiotics that were tested in the study. Each antibiotic has a gradient of eight
concentrations, which were made by two-fold dilutions, including the CLSI susceptibility
breakpoint, four concentrations below the breakpoint, and three concentrations
above the breakpoint. All concentrations are in μg/mL.

| Augmentin | | | | | | |
|---|---|---|---|---|---|---|
| 2:1 | | | | | | |
| (Amoxicillin: | | | | | | |
| Clavulanate) | Cefuroxime | Nitrofurautoin | Ceftriaxone | Meropenem | Cefoxitin | Levofloxacin |
| 1/0.5 | 0.5 | 2 | 0.125 | 0.25 | 1 | 0.125 |
| 2/1 | 1 | 4 | 0.25 | 0.5 | 2 | 0.25 |
| 4/2 | 2 | 8 | 0.5 | 4 | 4 | 0.5 |
| 8/4 | 4 | 16 | 1 | 2 | 8 | 1 |
| 16/8 | 8 | 32 | 2 | 4 | 16 | 2 |
| 32/36 | 16 | 64 | 4 | 8 | 32 | 4 |
| 64/32 | 32 | 128 | 8 | 16 | 64 | 8 |
| 128/64 | 64 | 256 | 16 | 32 | 128 | 16 |

| Augmentin | | | | | |
|---|---|---|---|---|---|
| 2:1 | | | | | |
| (Amoxicillin: | | | | | |
| Clavulanate) | Cefazolin | Ampicillin | Clindamycin | Ciprofloxacin | Doxycycline |
| 1/0.5 | 0.25 | 1 | 0.0625 | 0.0039 | 2 |
| 2/1 | 0.5 | 2 | 0.125 | 0.0078 | 4 |
| 4/2 | 1 | 4 | 0.25 | 0.0156 | 8 |
| 8/4 | 2 | 8 | 0.5 | 0.0313 | 16 |
| 16/8 | 4 | 16 | 1 | 0.0625 | 32 |
| 32/36 | 8 | 32 | 2 | 0.125 | 64 |
| 64/32 | 16 | 64 | 4 | 0.25 | 128 |
| 128/64 | 32 | 128 | 8 | 0.5 | 256 |

TABLE 2
The BLAST assay list of reagents including BHI as Buffer
A, HPG as Buffer B, PBS as Buffer W, and the click-
chemistry mix plus dye as Buffers C1 and C2 mix.

| Content | Label |
|---|---|
| Acclimatization media (Brain Heart Infusion) | Buffer A |
| Labeling mix (HPG) | Buffer B |
| Wash buffer (PBS) | Buffer W |
| Two component tag mix (Click-chemistry mix plus dye) | Buffer C1 |
| | Buffer C2 |

### Preparation of Buffers for the BLAST Assay

The acclimatization media (Buffer A) is Brain Heart Infusion liquid media. Buffer B is the labeling buffer and was made by dissolving HPG in sterile nuclease-free water at 3.9 mg/mL (stock solution). The tagging buffer (Buffer C1) contains the fluorescent dye used for tagging labeled proteins in the click chemistry reaction and is composed of 100 μM CuSO4, 25 μM AZDye 488 Azide, and 25 μM BTTAA in HEPES buffered saline with 0.1% Tween20. Buffer C2 is used as a reducing agent in the click chemistry reaction, and it contains 4 mM sodium ascorbate in HEPES buffered saline with 0.1% Tween20. The wash buffer (Buffer W) is used for removing the excess and unbound HPG and fluorescent dye during the click chemistry reaction and is made up of PBS, pH 7.4 with 0.1% Tween20.

### The BLAST Labeling Response as a Function of Bacterial Concentration

A study was performed to evaluate the BLAST labeling response as a function of bacterial concentration in the absence of antibiotics using a representative Gram-negative (E. coli ATCC 25922) and Gram-positive (S. aureus ATCC 29213) strain. Test inocula ranged from 1.5×10¹ to 1.5×10⁸ CFU/mL, and each condition was tested in triplicate. The full BLAST assay was performed without antibiotics to measure the effects of bacterial concentration on protein labeling and subsequent fluorescent detection as described below.

The assay was performed on a 0.2 μm 96-well Supor (polyethersulfone) filtration plate (Cytiva, Cat. #8019) which allowed the pellet to be washed by filtration during the assay. On each filtration plate (FIG. 6), some wells were used for sterility control, and each test was performed in triplicate. Sterility control wells did not have bacterial samples, and background control wells did not have the labelling buffer. As illustrated in the workflow (FIGS. 1 and 4), colonies were picked from an overnight-grown blood agar plate and used to inoculate 10 mL of Buffer A in 8 different 50 mL culture tubes at varying inocula (1.5×10¹ CFU/mL, 1.5×10² CFU/mL, 1.5×10³ CFU/mL, 1.5×10⁴ CFU/mL, 1.5×10⁵ CFU/mL, 1.5×10⁶ CFU/mL, 1.5× 10⁷ CFU/mL, and 1.5×10⁸ CFU/mL). The cultures were incubated at 37° C. for 45 minutes with shaking at 250 rpm (acclimatization step). The filtration plate was prepared for the assay by sealing at the bottom using a plate seal to prevent any leaks during the assay. Buffer A was added to all the wells at 65 μL/well. After 45 minutes of acclimatization, 65 μL of each bacterial culture was added to the respective test wells and background control wells. The sterility control wells received 65 μL of buffer A instead of bacterial culture. The plate was sealed at the top using the AeraSeal™ plate adhesive and then incubated at 37° C. for 2 hours with shaking at 250 rpm. To begin the labeling process, the AeraSeal™ plate adhesive was removed from the plate, and then 20 μL of buffer B (0.52 mg/mL being the final concentration) was added to each test well and sterility control well. Background control wells received 20 μL of sterile water instead of Buffer B. The plate was sealed at the top using a fresh AeraSeal™ plate adhesive and then incubated at 37° C. for 1.5 hours. To prepare for filtration and washing, the top AeraSeal™ plate adhesive and the bottom seal were both removed from the filtration plate. The liquid was filtered out using a vacuum pump and manifold with the pressure set at 20-25 mm Hg, and the filtrate was discarded. The plate was washed once by adding 300 μL of Buffer W into each well and filtering by vacuum as described above. The filtrate was discarded, and the filtration plate was sealed at the bottom using a fresh plate seal as described earlier. Buffer C1 and C2 were mixed in a ratio of 1:1 to form the tagging buffer mix, which was added to each well of the filtration plate at 100 μL/well. The plate was sealed at the top using a fresh AeraSeal™ plate adhesive, followed by incubation at 37° C. for 30 minutes with shaking at 250 rpm. The top and bottom seals were then removed from the filtration plate to prepare for washing. The liquid was drained, and the plate was washed three times using the vacuum filtration system as described above. The plate was wiped to remove any liquid drops before reading the fluorescent signal in a SpectraMax M2 plate reader (Molecular Devices) at the excitation and emission wavelengths of 484 nm and 524 nm, respectively. The Relative Fluorescence Units (RFU) readings were recorded for each well. The labeling response was assessed by comparing fluorescence signals from test samples to those of background controls, enabling evaluation of signal strength across the inocula range.

### Performing the BLAST Assay Using Isolates

The BLAST assay was performed to determine the MIC values for E. coli ATCC 25922 with 11 antibiotics (nitrofurantoin, cefazolin, doxycycline, levofloxacin, meropenem, ceftriaxone, ciprofloxacin, cefuroxime, amoxicillin/clavulanate, ampicillin, and cefoxitin), and S. aureus ATCC 29213 against 9 antibiotics (nitrofurantoin, doxycycline, levofloxacin, meropenem, ceftriaxone, ciprofloxacin, amoxicillin/clavulanate, ampicillin, and cefoxitin). The assay was performed using a modification of the method described for BLAST labeling response as a function of bacterial concentration. Eight concentrations (TABLE 1) of each antibiotic were tested in triplicate. The eight concentrations, which were made by two-fold dilutions included the CLSI susceptible breakpoint, four concentrations below the breakpoint, and three concentrations above the breakpoint. On each filtration plate, two wells were used for sterility control, two wells for background control and four wells for positive control. All controls did not have antibiotics, the sterility control did not have the bacterial sample, and the background control did not have the labelling buffer.

Colonies were picked from an overnight-grown blood agar plate and used to inoculate 10 mL of Buffer A in a 50 mL culture tube at 5.0×10⁶ CFU/mL. The culture was incubated at 37° C. for 45 minutes with shaking at 250 rpm. The filtration plate was sealed at the bottom using a plate seal to prevent any leaks during the assay. Antibiotic gradients (Table 1) were prepared in the filtration plate by adding 65 μL of each drug concentration (diluted in buffer A) into the respective test wells. All control wells received 65 μL of buffer A instead of drugs. After 45 minutes of acclimatization, 65 μL of the bacterial culture was added to each test well of the filtration plate containing a drug and to the positive and background control wells. The sterility control wells received 65 μL of buffer A instead of culture. The plate was sealed at the top using the AeraSeal™ plate adhesive and then incubated at 37° C. for 2 hours with shaking at 250 rpm. To begin the labeling process, the AeraSeal™ plate adhesive was removed from the plate and then 20 μL of buffer B (0.52 mg/mL final concentration) was added to each test well, the positive control wells, and the sterility control wells. Background control wells received 20 μL of sterile water instead of Buffer B. The plate was sealed at the top using a fresh AeraSeal™ plate adhesive and then incubated at 37° C. for 1.5 hours. The plate was washed, processed using the tagging buffer (Buffer C1 and C2 mix) and fluorescence signal recorded using the method described for BLAST labeling response as a function of bacterial concentration. Using the Relative Fluorescence Units (RFU) readings, BLAST MICs were determined as described below.

### Blast Mic Determination

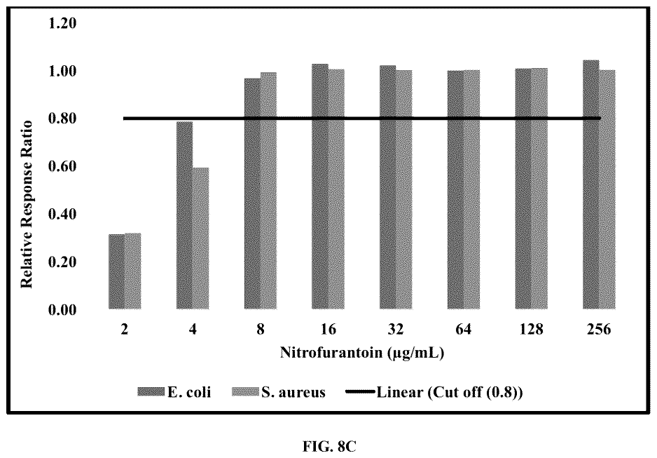

With reference to FIGS. 8A-8C, plotting the antibiotic dosage response of the fluorescence typically yields a curve, and in cases where the antibiotic is effective against the bacteria, in the range of concentrations tested, the signal is substantially decreased. Presented herein is an objective mathematical technique for generating a value that is functionally similar to a minimum inhibitory concentration as determined by the reference method. For each antibiotic, the BLAST MIC value was determined by first, calculating the Relative Response Ratio (RRR) for each antibiotic concentration, as described in Formula 1 below, and then comparing the eight RRR values to the BLAST cut-off point of 0.8.

$$RRR = \frac{Average Positive Control Value - Average Sample Value}{Average Positive Control Value - Background Value} \qquad ( 1 )$$

The BLAST MIC value is the lowest antibiotic concentration at which the RRR equals or surpasses the BLAST cut-off point. BLAST MICs were determined for each sample and then compared to the corresponding CLSI QC range.

### Comparing BLAST to the Reference Broth Microdilution Method

Four bacterial strains (E. coli ATCC 25922, A. baumannii NCTC 13304, E. coli LSI 770, and K. pneumoniae ATCC 700603) were tested by both BLAST and rBMD to determine their MIC values against nine antibiotics (amoxicillin/clavulanate, cefuroxime, nitrofurantoin, ceftriaxone, meropenem, cefoxitin, levofloxacin, cefazolin, and ampicillin). The BLAST test was performed using isolates as described earlier (Performing the BLAST assay using isolates) in this document, but the assay culture was started at 1×10⁵ CFU/mL. The rBMD test was performed using the standard protocol [19,20]. Each test was performed in triplicate, and the resultant BLAST MICs were compared to their corresponding rBMD MICs.

### Performing the BLAST Assay Using Bacteria-Contrived Urine Culture

The BLAST assay was performed using bacteria-contrived urine culture to determine the MICs for E. coli ATCC 25922 and E. coli LSI 770 against doxycycline, levofloxacin, and cefazolin, and the MICs for S. aureus ATCC 29213 against doxycycline and levofloxacin. The assay was performed using a modification of the method described for BLAST labeling response as a function of bacterial concentration. Sterile human pooled urine samples were batch-tested to rule out bacterial contamination and then separately inoculated with colonies of the listed isolates which were picked from overnight blood agar plates. The bacteria-inoculated urine samples were incubated overnight at 37° C. with shaking at 200 rpm in separate culture tubes. The overnight grown urine cultures were aliquoted into two batches. The first batch of samples was centrifuged at 2800×g for 10 minutes, and the pellet was used to prepare an inoculum of 5.0× 10⁵ CFU/mL to start the BLAST assay using the method described earlier for testing isolates. Each sample was tested against eight different concentrations (TABLE 1) of doxycycline, levofloxacin, and cefazolin, and the fluorescence signals were used to determine BLAST MICs using the formula described for BLAST MIC determination. The second batch of overnight-grown bacteria-contrived urine cultures was plated on agar plates and cultured overnight at 37° C. The isolates from the overnight plates were then tested by BLAST against eight different concentrations of doxycycline, levofloxacin, and cefazolin (TABLE 1) at 5.0×10⁵ CFU/mL and by the rBMD method (CLSI M07, 2025) to determine MICs. Each test was performed in triplicate, and for each bacteria-contrived urine sample tested by BLAST, corresponding isolates were also tested by both BLAST and the rBMD method, resulting in three MIC values (the BLAST MIC from the bacteria-contrived urine test, the BLAST MIC from isolated colonies test, and the rBMD MIC). Each set of three corresponding MICs were compared to each other.

### Results

### BLAST Labeling Response as a Function of Bacterial Concentration

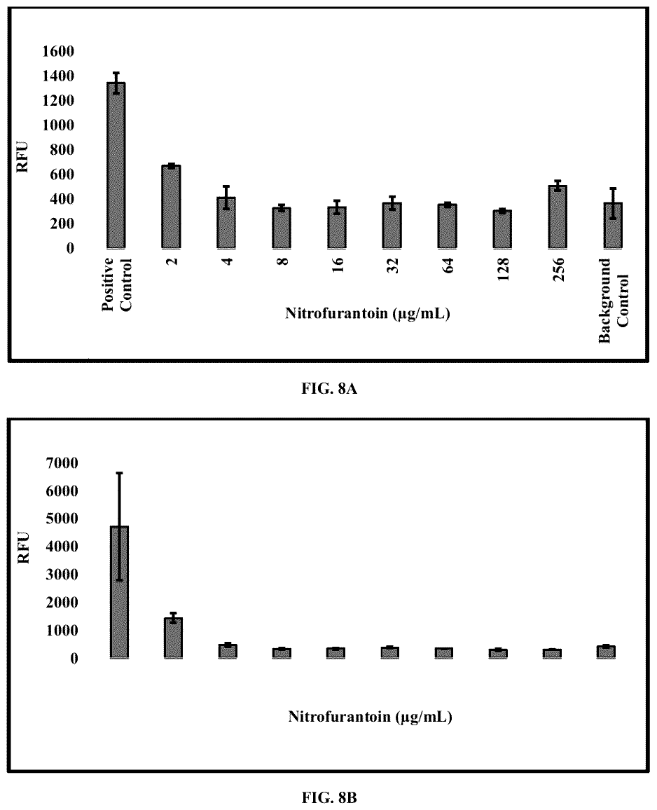

The BLAST detection data for the two reference strains, E. coli ATCC 25922 and S. aureus ATCC 29213, is shown in FIG. 7A-7B. The starting inocula concentrations varied from 1.5×10¹ to 1.5×10⁸ CFU/mL and the BLAST detection range was determined by comparing the signal difference between the positive and the background control samples. Results for both E. coli ATCC 25922 (FIG. 7A) and S. aureus ATCC 29213 (FIG. 7B) show a clear difference between positive and background signals when the starting bacterial concentration ranged from 1.5×10⁵ to 1.5×10⁸ CFU/mL for both strains. Each test was performed in triplicate, and error bars represent the standard deviation.

### Performing the BLAST Assay Using Isolates

The BLAST assay was performed using isolated colonies to determine the MICs for E. coli ATCC 25922, and S. aureus ATCC 29213 against 11 and 9 antibiotics respectively. Results in FIGS. 8A-8C demonstrate the process of using BLAST to determine the nitrofurantoin MIC values for E. coli ATCC 25922 and S. aureus ATCC 29213, as an example. As presented in these Figures, the BLAST fluorescent responses (FIGS. 8A and 8B) were converted into the RRR (FIG. 8C) to determine the BLAST MIC values. The experiments were repeated in triplicate and error bars in FIGS. 8A and 8B represent the standard deviation. As presented in FIG. 8C, the BLAST MIC is the lowest antibiotic concentration at which the RRR equals or surpasses the BLAST cut-off point (0.8). Based on the RRR values, the BLAST MIC values for both strains against nitrofurantoin were determined to be 8 μg/mL. The nitrofurantoin CLSI QC range is 4-16 μg/mL and 8-32 μg/mL for E. coli ATCC 25922 and S. aureus ATCC 29213 respectively. This shows that both BLAST MICs were within the CLSI QC range. The MIC values for each strain against the antibiotics were determined using the same method, and the results in Table 3 show that all the MIC values for E. coli ATCC 25922 against the 11 antibiotics are within the CLSI QC range. These results also show that all MIC values for S. aureus ATCC 29213 against the 9 antibiotics fall within the CLSI QC range except doxycycline.

TABLE 3
A summary of results from the BLAST assay using isolates. The
BLAST assay was used to determine MICs for E. coli ATCC 25922
and S. aureus ATCC 29213 against 11 and 9 antimicrobial agents
respectively. Bacterial isolates were tested at a starting
inoculum of 5 × 10⁶ CFU/mL. Each BLAST MIC value was compared
to the corresponding CLSI QC range, and all the values match
except, S. aureus ATCC 29213 against doxycycline.

| | | CLSI | | |
|---|---|---|---|---|
| | | Breakpoints¹ | MIC (μg/mL) | |
| Bacterial | | (μg/mL) | | CLSI QC |
| Strain | Antibiotic | (≤S, I, ≥R) | BLAST² | Range¹ |
| E. coli | Nitrofurantoin | 32, 64, 128 | 4 | 4.0-16.0 |
| ATCC | Meropenem | 1, 2, 4 | 0.016 | 0.008-0.06 |
| 25922 | Ceftriaxone | 1, 2, 4 | 0.06 | 0.03-0.12 |
| | Ciprofloxacin | 0.25, 0.5, 1 | 0.016 | 0.004-0.016 |
| | Cefuroxime | 4, 8-16, 32 | 4 | 2.0-8.0 |
| | Amoxicillin/ | 8/4, 16/8, | 2/1 | 2/1-8/4 |
| | Clavulanate (2:1) | 32/16 | | |
| | Ampicillin | 8, 16, 32 | 4 | 2.0-8.0 |
| | Cefoxitin | 8, 16, 32 | 4 | 2.0-8.0 |
| | Doxycycline | 4, 8, 16 | 0.5 | 0.5-2.0 |
| | Levofloxacin | 0.5, 1, 2 | 0.03 | 0.008-0.06 |
| | Cefazolin | 16, —, 32 | 2 | 1.0-4.0 |
| S. aureus | Nitrofurantoin | 32, 64, 128 | 8 | 8.0-32.0 |
| ATCC | Meropenem | — | 0.06 | 0.03-0.12 |
| 29213 | Ceftriaxone | — | 1 | 1.0-8.0 |
| | Ciprofloxacin | 1, 2, 4 | 0.25 | 0.12-0.5 |
| | Amoxicillin/ | — | 0.12/0.06 | 0.12/0.06- |
| | Clavulanate 2:1 | | | 0.5/0.25 |
| | Ampicillin | — | 0.5 | 0.5-2 |
| | Cefoxitin | 4, —, 8 | | 1.0-4.0 |
| | Doxycycline | 4, 8, 16 | 0.016 | 0.12-0.5 |
| | Levofloxacin | 1, 2, 4 | 0.25 | 0.06-0.5 |

¹CLSI M100-Ed35
²Geometric Mean MIC (rounded to nearest 2-fold dilution; n = 3)

Comparing the BLAST Assay to the rBMD Method

The BLAST assay was compared to the rBMD method by determining the MIC values for E. coli ATCC 25922, A. baumannii NCTC 13304, E. coli LSI 770, and K. pneumoniae ATCC 700603 against 9 antibiotics using both methods. The BLAST and rBMD assays were performed in triplicate and the results are presented in Table 4. The data shows that all BLAST MICs for E. coli ATCC 25922 against the 9 antibiotics were comparable to the corresponding rBMD MICs. All MICs were determined to be within +1 dilution except meropenem and cefoxitin. However, both BLAST and rBMD MIC values for meropenem and cefoxitin are within the CLSI QC range despite the 2-dilution difference. All the BLAST MICs for A. baumannii NCTC 13304 and E. coli LSI 770 against the 9 antibiotics were comparable to the corresponding rBMD MICs and within +1 dilution. BLAST MICs for K. pneumoniae ATCC 700603 against the 9 antibiotics agree with all the corresponding rBMD MICs within +1 dilution except for ceftriaxone and meropenem which were different by two-dilutions. There was no established CLSI QC range for K. pneumoniae ATCC 700603 against ceftriaxone and meropenem for comparison. All the interpreted susceptibilities from both the BLAST method and standard methods are similar except E. coli ATCC 25922 against cefuroxime which showed BLAST as susceptible and rBMD as intermediate (S, I).

TABLE 4
Comparing BLAST to the rBMD method. Four strains were each tested by both
BLAST and rBMD to determine MIC values against nine antibiotics. Each BLAST test was
performed at a starting inoculum of 1.0 × 10⁵ CFU/mL and the resultant BLAST MICs were
compared to their corresponding rBMD MIC values. All BLAST MIC values agreed (±1 dilution)
with their corresponding rBMD MICs except for E. coli ATCC 25922 against meropenem and
cefoxitin, and K. pneumoniae ATCC 700603 against ceftriaxone and meropenem. All the interpreted
susceptibilities from both the BLAST method and standard methods are similar except E. coli ATCC
25922 against cefuroxime which showed BLAST as susceptible and rBMD as intermediate (S, I).

| | MIC¹ (μg/ml) | | | | | | | | | | | |
|---|---|---|---|---|---|---|---|---|---|---|---|---|
| | E. coli | | | A. baumannii | | | E. coli | | | K. pneumoniae | | |
| Antibiotic | ATCC 25922 | | | NCTC 13304 | | | LSI 770 | | | ATCC 700603 | | |
| Amoxicillin/ | BLAST | rBMD | S, I, R | BLAST | rBMD | S, I, R | BLAST | rBMD | S, I, R | BLAST | rBMD | S, I, R |
| Clavulanate (2:1) | 4/2 | 8/4 | S | 256 | >256 | N/A | 32 | 64 | R | 16 | 16 | I |
| Cefuroxime² | 4 | 8 | S, I | >256 | >256 | N/A | >256 | >256 | R | >64 | 64 | R |
| Nitrofurantoin | 8 | 8 | S | 128 | 256 | N/A | 16 | 16 | S | 128 | 64 | R |
| Ceftriaxone | 0.125 | 0.0625 | S | 256 | >256 | R | >256 | >256 | R | 16 | 4 | R |
| Meropenem | 0.0625 | 0.015 | S | 32 | 32 | R | 0.03 | 0.03 | S | 0.0625 | 0.015 | S |
| Cefoxitin³ | 8 | 2 | S | >256 | >256 | N/A | 128 | 64 | R | 128 | 64 | R |
| Levofloxacin | 0.015 | 0.008 | S | 1 | 1 | S | 32 | 16 | R | 0.25 | 0.5 | S |
| Cefazolin | 8 | 2 | S | >256 | >256 | N/A | >256 | >256 | R | >32 | 128 | R |
| Ampicillin | 2 | 4 | S | >256 | >256 | N/A | >256 | >256 | R | >256 | >256 | R |

S = Susceptible, I = Intermediate, R = Resistant.
¹Mean MIC (rounded to nearest 2-fold dilution; n = 3).
²Used oral breakpoints for enteric species.
³Used uncomplicated UTI breakpoints for enteric species.

### Performing the BLAST Assay Using Bacteria-Contrived Urine Culture

Bacteria-contrived urine cultures of E. coli ATCC 25922, S. aureus ATCC 29213, and E. coli LSI 770 were tested in the BLAST assay to determine MICs against doxycycline, levofloxacin, and cefazolin (S. aureus ATCC 29213 was not tested with cefazolin). E. coli LSI 770 (CTX-M-15, OXA-1, and OXA-30) is a multiple-drug-resistant isolate that was used in the assay to compare with susceptible strains [21,22]. Isolates were also generated from the same bacteria-contrived urine cultures and tested by both BLAST and rBMD. This resulted in three sets of MICs for each tested sample, the BLAST MIC for the bacteria-contrived urine culture test, the BLAST MIC for tested isolates, and the rBMD MIC. All the BLAST and rBMD tests were performed in triplicate and the results are presented in TABLE 5, below. Results show that samples gave comparable BLAST MICs when tested either as bacteria-contrived urine cultures or as isolates. Similarly, BLAST MIC values were comparable to their corresponding rBMD MICs except for two variations in the case of E. coli ATCC 25922 against doxycycline, and S. aureus ATCC 29213 against doxycycline. These two BLAST MICs differed from rBMD MICs by two dilutions.

TABLE 5
A summary of the BLAST assay data using bacteria-contrived urin esamples.
The BLAST assay was performed to determine MICs for E. coli ATCC 25922 and E. coli
LSI 770 against doxycycline, levofloxacin, and cefazolin and S. aureus ATCC 29213 against
doxycycline and levofloxacin using both bacteria-contrived urine samples and isolates at a starting
inoculum of 5 × 10⁵ CFU/mL. For each bacteria-contrived urine sample tested by BLAST,
corresponding isolates were also tested by the normal BLAST method and the rBMD method
resulting in three MIC values (the BLAST MIC from bacteria-contrived sample test, the BLAST
MIC from isolates, and the rBMD MIC). Each set of three corresponding MICs were compared to
each other. All the BLAST MIC values were comparable to rBMD MICs except for E. coli ATCC
25922 isolates against doxycycline and S. aureus ATCC 29213 against doxycycline.

| | | | | MIC | | | | |
|---|---|---|---|---|---|---|---|---|
| | | | | (μg/mL) | | | Dilution Difference | |
| | | | CLSI | BLAST² | | | (BLAST-rBMD) | |
| | | | Breakpoints¹ | Bacteria- | | | Bacteria- | |
| Bacteria | | | (μg/mL) | contrived | Isolated | | contrived | Isolated |
| Strain | Antibiotic | S, I, R | (≤S, I, ≥R) | Urine | Colonies | rBMD² | Urine | Colonies |
| E. coli | Doxycycline | S | 4, 8, 16 | 0.25 | 0.125 | 0.5 | −1 | −2 |
| ATCC | Levofloxacin | | 0.5, 1, 2 | 0.016 | 0.016 | 0.016 | 0 | 0 |
| 25922 | Cefazolin | S | 16, -, 32 | | 4 | 2 | −1 | +1 |
| S. aureus | Doxycycline | S | 4, 8, 16 | 0.03 | 0.03 | 0.12 | −2 | −2 |
| ATCC | | | | | | | | |
| 29213 | Levofloxacin | S | 1, 2, 4 | 0.25 | 0.25 | 0.25 | 0 | 0 |
| E. coli | Doxycycline | R | | 64 | 64 | 64 | 0 | 0 |
| LSI 770 | Levofloxacin | R | | 32 | 32 | 32 | 0 | 0 |
| | Cefazolin | R | | >256 | >256 | >256 | 0 | 0 |

¹CLSI M100-Ed35 | ²Geometric Mean MIC (rounded to nearest 2-fold dilution; n = 3) | S = Susceptible, I = Intermediate, R = Resistant.

### Discussion

In the study described herein, the BLAST assay was used to test the antibiotic susceptibility of both Gram-negative and Gram-positive strains of bacteria known to cause UTI. Diverse classes of antibiotics that are expected to target different cellular processes were chosen for the test. Most of these antibiotics are commonly used or appropriate for the treatment of urinary tract infections. It is expected that the production of new proteins, a requirement for the BLAST assay, will be halted by antibiotics that are known to act by blocking translation. However, it was also observed that the other classes of antibiotics targeting cell wall or nucleic acid synthesis were effective in reducing labeling. This result is consistent with multiple studies showing that during periods of stress bacteria shut down protein synthesis as a viability-preserving bacterial stress response. MIC values were determined using BLAST and compared the values to the corresponding rBMD MICs and CLSI QC ranges that were available. All the BLAST MIC values for E. coli ATCC 25922 against the eleven antibiotics were within the CLSI QC range. In the case of S. aureus ATCC 29213, all the BLAST MIC values were within the CLSI QC range except for doxycycline.

When actively metabolizing bacteria are exposed to the ncAA compound HPG, the amount of label and subsequent fluorescent tagging can vary by strain and bacterial concentration. For each sample tested, the average fluorescence of positive control wells is used to normalize the values. As described in the methods, the BLAST MIC value is determined by first, calculating the Relative Response Ratio (RRR) for each antibiotic concentration and then comparing the eight RRR values to the cut-off point of 0.8. This cutoff value corresponds to roughly 80% inhibition of signal, by the antibiotic, accounting for background. The RRR calculation thus accounts for labeling efficiency and corrects for background, typically returning a value between 0.8 to 1 for cases where the antibiotic completely suppresses new protein synthesis, and approaches zero when the antibiotic concentration is ineffective at inhibiting the bacterial activity. The BLAST MIC value presented is the lowest antibiotic concentration at which the RRR equals or surpasses the BLAST cut-off point. For this study, a practical cut-off point of 0.8 was set, which represents a substantial reduction in new protein biosynthesis. This cut-off point has worked successfully in matching the BLAST results with the rBMD, but can be further calibrated with data from clinical studies.

By varying the starting bacterial concentration, the study was able to demonstrate that the BLAST methodology works over a wide range of starting inoculum concentrations (10⁵ CFU/mL to >10⁸ CFU/mL), which are also in the range of bacterial concentrations commonly encountered in active urinary tract infections [23]. The BLAST system was tested at a broader range of bacterial concentration (1.0×10⁵ CFU/mL to 5.0×10⁶ CFU/mL) as part of the methods development because the BLAST assay is optimizable for direct urine testing. A typical positive urine culture is defined as having organisms in a concentration≥1.0×10⁵ CFU/mL [23]. As such, samples at 1.0×10⁵ CFU/mL (Table 4), 5.0×10⁵ CFU/mL (Table 5), and 5.0×10⁶ CFU/mL (TABLE 3) were tested. The same strains were tested multiple times at different concentrations within this range, and the same MIC results were obtained. In general, the technique does not show a large effect of high inoculum for the antibiotics tested here, possibly because the bacteria are not required to grow to saturation for detection of turbidity, and inactive bacterial mass do not contribute much to the signal. Furthermore, the performance of BLAST was evaluated in comparison to the reference broth microdilution method and in general, there was a high degree of correlation between the BLAST MICs and the rBMD MICs. This correlation includes (critically) the results for E. coli LSI 770, the strain that exhibits multiple drug resistance and has MIC values at the higher end of the test ranges.

In a clinical application, an AST assay functions to rule out antibiotics that will not kill the strain and identifies the best options for treatment. The BLAST assay was performed using both isolates and bacteria-contrived urine cultures, and in both cases the assay was accomplished in 6 hours with comparable BLAST MICs. Both sets of MIC values matched the corresponding rBMD MIC values for most of the tested samples.

Rapid AST is essential for guiding healthcare professionals in determining effective antibiotic treatments, combating the rise of drug-resistant bacteria, and facilitating the introduction of new antibiotics. Traditional broth microdilution and related methods require many rounds of bacterial replication and sample processing steps, which hamper the development of direct-from-sample testing. By contrast, BLAST testing takes a significantly shorter duration than traditional AST methods and does not require extensive cell replication. In addition, the BLAST assay detects proteins without requiring extensive sample processing. Since BLAST is based on measurement of newly synthesized proteins, it differs from other AST systems that rely on periodic assessment of culture growth for accurate MIC results and can be applied to both bactericidal and bacteriostatic antibiotics. The BLAST assay is performed in a 96-well filtration plate which makes the system scalable and lends itself to automation and liquid handling. BLAST testing of bacterial isolates is straightforward, and bacteria-contrived urine cultures have been demonstrated to be a qualified basis for direct-from-sample techniques using BLAST.

All literature and similar material cited in this application, including, but not limited to, patents, patent applications, articles, books, treatises, and web pages, regardless of the format of such literature and similar materials, are expressly incorporated by reference in their entirety. Should one or more of the incorporated references and similar materials differ from or contradict this application, including but not limited to defined terms, term usage, described techniques, or the like, this application controls.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about”, if or when used throughout this specification describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

There may be many alternate ways to implement the disclosed technology. Various functions and elements described herein may be partitioned differently from those shown without departing from the scope of the disclosed technology. Generic principles defined herein may be applied to other implementations. Different numbers of a given module or unit may be employed, a different type or types of a given module or unit may be employed, a given module or unit may be added, or a given module or unit may be omitted.

Regarding this disclosure, the term “a plurality of” refers to two or more than two. Unless otherwise clearly defined, orientation or positional relations indicated by terms such as “upper” and “lower” are based on the orientation or positional relations as shown in the Figures, only for facilitating description of the disclosed technology and simplifying the description, rather than indicating or implying that the referred devices or elements must be in a particular orientation or constructed or operated in the particular orientation, and therefore they should not be construed as limiting the disclosed technology. The terms: “connected”, “mounted”, “fixed”, etc. should be understood in a broad sense. For example, “connected” may be a fixed connection, a detachable connection, or an integral connection, a direct connection, or an indirect connection through an intermediate medium. For an ordinary skilled in the art, the specific meaning of the above terms in the disclosed technology may be understood according to specific circumstances.

It should be appreciated that all combinations of the foregoing concepts and additional concepts discussed in greater detail herein (provided such concepts are not mutually inconsistent) are contemplated as being part of the disclosed technology. In particular, all combinations of claimed subject matter appearing at the end of this disclosure are contemplated as being part of the technology disclosed herein. While the disclosed technology has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed technology in its broader aspects is not limited to any of the specific details, representative devices and methods, and/or illustrative examples shown and described. Accordingly, departures may be made from such details without departing from the spirit or scope of the general inventive concept.

The following references form part of the specification of the present application and each reference is incorporated by reference herein, in its entirety, for all purposes.
[1] Geerlings S E. Clinical presentations and epidemiology of urinary tract infections. Microbiol Spectr 2016; 4 (5). https://doi.org/10.1128/microbiolspec. UTI-0002-2012.
[2] Infectious Diseases Society of America. Guideline on management and treatment of complicated urinary tract infections: Executive summary. 2025. Published Jul. 17, 2025.
[3] Sabih A, Leslie S W. Complicated urinary tract infections. StatPearls. StatPearls Publishing; 2025. https://www.ncbi.nlm.nih.gov/books/NBK436013/.
[4] Zilberberg M D, Nathanson B H, Sulham K, Shorr A F. Descriptive epidemiology and outcomes of hospitalizations with complicated urinary tract infections in the United States. Open Forum Infect Dis 2022; 9 (1). https://doi.org/10.1093/ofid/ofab591.
[5] Mandal P K, Biswas A K, Choi K, Pal U K. Methods for rapid detection of foodborne pathogens: an overview. Am J Food Technol 2011; 6:87-10².
[6] Syal K, Mo M, Yu H, Iriya R, Jing W, Guodong S, et al. Current and emerging techniques for antibiotic susceptibility tests. Theranostics 2017; 7 (7): 1795-805. https://doi.org/10.7150/thno.19217.
[7] Law J W F, Ab Mutalib N S, Chan K G, Lee L H. Rapid methods for the detection of foodborne bacterial pathogens: principles, applications, advantages and limitations. Front Microbiol 2014; 5:770. https://doi.org/10.3389/fmicb.2014.00770.
[8] Wu W, Cai G, Liu Y, Suo Y, Zhang B, Jin W, et al. Direct single-cell antimicrobial susceptibility testing of Escherichia coli in urine using a ready-to-use 3D microwell array chip. Lab Chip 2023; 10:2399-410. https://doi.org/10.1039/D2LC01095J.
[9] Bock L J, Spencer D C, Martin B K, Daniels C N, Dyball X, Hind C K, et al. Rapid impedance-based antimicrobial susceptibility testing (iFAST) of enterobacterales in urinary tract infections. J Infect 2025; 91 (2): 106549. http://doi.org/10.1016/j. jinf.2025.106549.
[10] Wu W, Zhao Q, Cai G, Zhang B, Suo Y, Liu Y, et al. All-In-one Escherichia coli viability assay for multi-dimensional detection of uncomplicated urinary tract infections. Anal Chem 2022; 94 (51): 17853-60. https://doi.org/10.1021/acs. analchem.2c03604.
[11] Wu W, Zhang B, Yin W, Xia L, Suo Y, Cai G, et al. Enzymatic antimicrobial susceptibility testing with bacteria identification in 30 min. Anal Chem 2023; 95 (44): 16426-32. https://doi.org/10.1021/acs.analchem.3c04316.
[12] Wu W, Suo Y, Zhao Q, Cai G, Liu Y, Jin W, et al. Inoculum size-insensitive susceptibility determination of urine sample based on in-situ measurement of inducible enzyme activity after 20 min of antibiotic exposure. Anal Chim Acta 2023; 1282:341858. https://doi.org/10.1016/j.aca.2023.341858.
[13] Grammel M, Hang H C. Chemical reporters for biological discovery. Nat Chem Biol 2013; 9:475-84. https://doi.org/10.1038/nchembio.1296.
[14] Rangan K J, Yang Y Y, Charron G, Hang H C. Rapid visualization and large-scale profiling of bacterial lipoproteins with chemical reporters. J Am Chem Soc 2010; 132:10628-9. https://doi.org/10.1021/ja101387b.
[15] Link A J, Tirrell D A. Cell surface labeling of Escherichia coli via copper (I)-catalyzed [3+2] cycloaddition. J Am Chem Soc 2003; 125:11164-5. https://doi.org/10.1021/ja036765z.
[16] Sherratt A R, Rouleau Y, Luebbert C, Strmiskova M, Veres T, Bidawid S, et al. Rapid screening and identification of living pathogenic organisms via optimized bioorthogonal non-canonical amino acid tagging. Cell Chem Biol 2017; 24 (8): 1048-55. https://doi.org/10.1016/j.chembiol.2017.06.016. e3.
[17] Atwal S, Giengkam S, Jaiyen Y, Feaga H A, Dworkin J, Salje J. Clickable methionine as a universal probe for labelling intracellular bacteria. J Microbiol Method 2020; 169:105812. https://doi.org/10.1016/j.mimet.2019.105812.
[18] Kennedy D C, Mckay C S, Legault M C B, Danielson D C, Blake J A, Pegoraro A F, et al. Cellular consequences of copper complexes used to catalyze bioorthogonal click reactions. J Am Chem Soc 2011; 133:17993-8001. https://doi.org/10.1021/ja2083027.
[19] Clinical and Laboratory Standards Institute. Methods for dilution antimicrobial susceptibility tests for bacteria that grow aerobically. 2024. 12th ed., CLSI standard M07.
[20] Clinical and Laboratory Standards Institute. Performance standards for antimicrobial susceptibility testing. 2025. 35th ed., CLSI supplement M100.
[21] Boyd D A, Mulvey M R. OXA-1 is OXA-30 is OXA-1. J Antimicrob Chemother 2006; 58 (1): 224-5. https://doi.org/10.1093/jac/dk1154.
[22] Markovska R, Schneider I, Keuleyan E, Bauernfeind A. Extended-spectrum β-lactamase (ESBL) CTX-M-15-producing Escherichia coli and Klebsiella pneumoniae in Sofia, Bulgaria. Clin Microbiol Infect 2004; 10 (8): 752-5. https://doi.org/10.1111/j.1469-0691.2004.00929.x.
[23] Kass E H. Pyelonephritis and bacteriuria. A major problem in preventive medicine. Ann Intern Med 1962; 56:46-53. https://doi.org/10.7326/0003-4819-56-1-46.

What is claimed:

1. A direct-from-sample antimicrobial susceptibility testing system, comprising:
(a) a biological sample taken directly from a patient, wherein the biological sample is processed to capture microorganisms present in the biological sample;
(b) an acclimatization medium into which the captured microorganisms are directly transferred to create a test sample, wherein the acclimatization medium is operative to activate protein biosynthesis in the microorganisms in the test sample after a predetermined period of time;
(c) a library of antimicrobials at predetermined concentrations to which the test sample is exposed for a predetermined period of time, wherein the exposure of the microorganisms in the test sample to the library of antimicrobials either kills or blocks protein biosynthesis in the microorganisms that are sensitive to one or more of the antimicrobials at one or more of the predetermined concentrations;
(d) a non-canonical amino acid, wherein the non-canonical amino acid is incorporated into newly biosynthesized proteins in the microorganisms that initially survive exposure to the antimicrobials thereby labeling the newly biosynthesized proteins; and
(e) a detectable tag configured to attach to the non-canonical amino acid labels in the newly biosynthesized proteins, wherein tagging the labeled proteins with the detectable tag creates an amount of detectable signal.

2. The system of claim 1, further comprising a system or device for detecting the signal.

3. The system of claim 1, further comprising a system or device for comparing the amount of detected signal in the test sample to a positive control,
(i) wherein the positive control is a test sample that has been labeled and tagged but has not been exposed to the library of antimicrobials,
(ii) wherein an observed absence of or a decrease in the amount of detectable signal relative to the positive control indicates susceptibility of the living microorganisms to one or more of the antimicrobials in the library of antimicrobials at one or more of the predetermined concentrations, and
(iii) wherein an observed signal that approaches or is equal to the value of the positive control indicates resistance of the living microorganisms to one or more of the antimicrobials in the library of antimicrobials at one or more of the predetermined concentrations.

4. The system of claim 1, wherein the patient is suspected of having an infection caused by one or more pathogenic microorganisms, wherein the suspected infection is a complicated urinary tract infection, and wherein the one or more pathogenic microorganisms are suspected of being present in the biological sample.

5. The system of claim 4, wherein the biological sample is derived from a bodily fluid or other bodily source.

6. The system of claim 5, wherein the bodily fluid is urine.

7. The system of claim 1, wherein the microorganisms include bacteria.

8. The system of claim 1, wherein the library of antimicrobials includes antibiotics.

9. The system of claim 1, wherein the non-canonical amino acid is an alkyne-modified non-canonical amino acid.

10. The system of claim 9, wherein the alkyne-modified non-canonical amino acid is L-Homopropargylglycine (HPG).

11. The system of claim 1, wherein the newly biosynthesized proteins include surface proteins, internal proteins, or a combination of surface proteins and internal proteins of the microorganisms in the test sample.

12. The system of claim 1, wherein the detectable tag is an azide-modified detection molecule that is reacted with the alkyne-modified non-canonical amino acid using click-chemistry.

13. The system of claim 12, wherein the azide-modified detection molecule is a biotinylated ligand, a fluorogenic or fluorescent azide probe, or a fluorogenic or fluorescent dye.

14. The system of claim 1, wherein the detectable signal is generated using a method that is fluorescence-based, enzyme-linked immunosorbent assay (ELISA)-based, cell-based, dot blot-based, or microscopy-based.

15. The system of claim 1, wherein the signal is quantifiable, and wherein a predetermined amount of detected signal is indicative of a minimum inhibitory concentration of antibiotic.

16. The system of claim 1, wherein the system is a high-throughput method executed on a multi-well plate or microplate, wherein the type of multi-well plate or microplate includes filtration plates, and wherein more than one type of antimicrobial may be tested on the multi-well plate or microplate.

17. A direct-from-sample method for determining antimicrobial susceptibility, comprising:
(a) obtaining a biological sample taken directly from a patient;
(b) processing the biological sample to capture microorganisms present in the biological sample;
(c) transferring the captured microorganisms directly into an acclimatization medium to create a test sample, wherein after a predetermined period of time, the acclimatization medium activates protein biosynthesis in the microorganisms in the test sample;
(d) exposing the test sample to a library of antimicrobials at predetermined concentrations for a predetermined period of time, wherein the exposure of the microorganisms in the test sample to the library of antimicrobials either kills or blocks protein biosynthesis in the microorganisms that are sensitive to one or more of the antimicrobials at one or more of the predetermined concentrations;
(e) labeling newly synthesized proteins in the microorganisms that initially survive exposure to the antimicrobials using a non-canonical amino acid that incorporates into the newly biosynthesized proteins;
(f) attaching a detectable tag to the non-canonical amino acids incorporated into the newly biosynthesized proteins, wherein tagging the labeled proteins with the detectable tag creates an amount of detectable signal; and
(g) detecting the amount of signal present in the test sample.

18. The method of claim 17, further comprising comparing the amount of detected signal in the test sample to a positive control,
(i) wherein the positive control is a test sample that has been labeled and tagged, but that has not been exposed to the library of antimicrobials,
(ii) wherein an observed absence of or a decrease in the amount of detectable signal relative to the positive control indicates susceptibility of the living microorganisms to one or more of the antimicrobials in the library of antimicrobials at one or more of the predetermined concentrations, and
(iii) wherein an observed signal that approaches or is equal to the value of the positive control indicates resistance of the living microorganisms to one or more of the antimicrobials in the library of antimicrobials at one or more of the predetermined concentrations.

19. The method of claim 17, wherein the patient is suspected of having an infection caused by one or more pathogenic microorganisms, and wherein the one or more pathogenic microorganisms are suspected of being present in the biological sample.

20. The method of claim 19, wherein the biological sample is urine, wherein the one or more pathogenic microorganisms include bacteria, and wherein the suspected infection is a complicated urinary tract infection.

21. The method of claim 17, wherein the non-canonical amino acid is an alkyne-modified non-canonical amino acid, and wherein the alkyne-modified non-canonical amino acid is L-Homopropargylglycine (HPG).

22. The method of claim 17, wherein the newly biosynthesized proteins include surface proteins, internal proteins, or a combination of surface proteins and internal proteins of the microorganisms in the test sample.

23. The method of claim 17, wherein the detectable tag is an azide-modified detection molecule that is reacted with the alkyne-modified non-canonical amino acid using click-chemistry, and wherein the azide-modified detection molecule is a biotinylated ligand, a fluorogenic or fluorescent azide probe, or a fluorogenic or fluorescent dye.

24. The method of claim 17, wherein the detectable signal is generated using a method that is fluorescence-based, enzyme-linked immunosorbent assay (ELISA)-based, cell-based, dot blot-based, or microscopy-based.

25. The method of claim 17, wherein the signal is quantifiable, and wherein a predetermined amount of detected signal is indicative of a minimum inhibitory concentration of antibiotic.

26. The method of claim 1, wherein the method is a high-throughput method executed on a multi-well plate or microplate, wherein the type of multi-well plate or microplate includes filtration plates, and wherein more than one type of antimicrobial may be tested on the multi-well plate or microplate.

27. A direct-from-sample method for determining antimicrobial susceptibility, comprising:
(a) obtaining a biological sample taken directly from a patient;
(b) processing the biological sample to capture microorganisms present in the biological sample;
(c) transferring the captured microorganisms directly into an acclimatization medium to create a test sample, wherein after a predetermined period of time, the acclimatization medium activates protein biosynthesis in the microorganisms in the test sample;
(d) exposing the test sample to a library of antimicrobials at predetermined concentrations for a predetermined period of time, wherein the exposure of the microorganisms in the test sample to the library of antimicrobials either kills or blocks protein biosynthesis in the microorganisms that are sensitive to one or more of the antimicrobials at one or more of the predetermined concentrations;
(e) labeling newly synthesized proteins in the microorganisms that initially survive exposure to the antimicrobials using a non-canonical amino acid that incorporates into the newly biosynthesized proteins;
(f) attaching a detectable tag to the non-canonical amino acids incorporated into the newly biosynthesized proteins, wherein tagging the labeled proteins with the detectable tag creates an amount of detectable signal;
(g) detecting the amount of signal present in the test sample;
(h) comparing the amount of detected signal in the test sample to a positive control,
(i) wherein the positive control is a test sample that has been labeled and tagged, but has not been exposed to the library of antimicrobials,
(ii) wherein an observed absence of or a decrease in the amount of detectable signal relative to the positive control indicates susceptibility of the living microorganisms to one or more of the antimicrobials in the library of antimicrobials at one or more of the predetermined concentrations, and
(iii) wherein an observed signal that approaches or is equal to the value of the positive control indicates resistance of the living microorganisms to one or more of the antimicrobials in the library of antimicrobials at one or more of the predetermined concentrations.

28. The method of claim 27, wherein the patient is suspected of having an infection caused by one or more pathogenic microorganisms, and wherein the one or more pathogenic microorganisms are suspected of being present in the biological sample.

29. The method of claim 28, wherein the biological sample is urine, wherein the one or more pathogenic microorganisms include bacteria, and wherein the suspected infection is a complicated urinary tract infection.

30. The method of claim 27, wherein the non-canonical amino acid is an alkyne-modified non-canonical amino acid, and wherein the alkyne-modified non-canonical amino acid is L-Homopropargylglycine (HPG).

31. The method of claim 27, wherein the newly biosynthesized proteins include surface proteins, internal proteins, or a combination of surface proteins and internal proteins of the microorganisms in the test sample.

32. The method of claim 28, wherein the detectable tag is an azide-modified detection molecule that is reacted with the alkyne-modified non-canonical amino acid using click-chemistry, and wherein the azide-modified detection molecule is a biotinylated ligand, a fluorogenic or fluorescent azide probe, or a fluorogenic or fluorescent dye.

33. The method of claim 29, wherein the detectable signal is generated using a method that is fluorescence-based, enzyme-linked immunosorbent assay (ELISA)-based, cell-based, dot blot-based, or microscopy-based.

34. The method of claim 27, wherein the signal is quantifiable, and wherein a predetermined amount of detected signal is indicative of a minimum inhibitory concentration of antimicrobial.

35. The method of claim 27, wherein the method is a high-throughput method executed on a multi-well plate or microplate, wherein the type of multi-well plate or microplate includes filtration plates, and wherein more than one type of antimicrobial may be tested on the multi-well plate or microplate.
